SEQUENCE LISTING (2 sequences)

SEQ ID NO: 1 - DNA, 13 bp, artificial
  Oligonucleotide sequence motif
gcaagcatcctgt

SEQ ID NO: 2 - DNA, 13 bp, artificial
  Oligonucleotide sequence motif
gcattggtattca

(19) United States
(12) Patent Application Publication
(10) Pub. No.: US 2020/0318103 A1
(43) Pub. Date: Oct. 8, 2020

(54) STEREODEFINED SUB-MOTIF OPTIMISATION METHODS

(71) Applicant: Roche Innovation Center Copenhagen A/S, Horsholm (DK)

(72) Inventors: Konrad Bleicher, Freiburg (DE); Henrik Frydenlund Hansen, Ringsted (DK); Troels Koch, KOBENHAVEN S. (DK); Nanna Albaek, Birkerød (DK); Erik Daa Funder, Hilleroed (DK)

(21) Appl. No.: 16/847,169

(22) Filed: Apr. 13, 2020

Related U.S. Application Data
(63) Continuation of application No. PCT/EP2018/077817, filed on Oct. 12, 2018.

(30) Foreign Application Priority Data
Oct. 13, 2017 (EP) 17196356.4
Aug. 17, 2018 (EP) 18189497.3

(51) Int. Cl. C12N 15/10; C40B 40/08

(52) U.S. Cl. CPC C12N 15/1072; C40B 40/08; C12N 2310/11; C12N 2330/31; C12N 2310/3231; C12N 2310/346; C12N 2310/315

(57) ABSTRACT

The present invention relates to methods for identifying improved stereodefined phosphorothioate oligonucleotide variants of antisense oligonucleotides utilising sub-libraries of partially stereodefined oligonucleotides. The methods allow for the efficient identification of stereodefined variants with improved properties, such as enhanced in vitro or in vivo activity, enhanced efficacy, enhanced specific activity, reduced toxicity, altered biodistribution, enhanced cellular or tissue uptake, and/or enhanced target specificity (reduced off-target effects).

### RELATED APPLICATIONS

This application is a Bypass continuation and claims priority to PCT/EP2018/077817 filed on Oct. 12, 2018, which claims priority to EP17196356.4 filed on Oct. 13, 2017 and EP 18189497.3 filed on Aug. 17, 2018. The entire contents of which are hereby incorporated by reference.

### FIELD OF INVENTION

The present invention relates to methods for identifying improved stereodefined phosphorothioate oligonucleotide variants of antisense oligonucleotides utilising sub-libraries of partially stereodefined oligonucleotides. The methods allow for the efficient identification of stereodefined variants with improved properties, such as enhanced in vitro or in vivo activity, enhanced efficacy, enhanced specific activity, reduced toxicity, altered biodistribution, enhanced cellular or tissue uptake, and/or enhanced target specificity (reduced off-target effects).

### BACKGROUND

Recently it has been established that production of stereodefined variants of a phosphorothioate antisense oligonucleotide can be used to create a phenomenal pharmacological diversity, and as with small molecule drug discovery paradigms, apparently small structural differences between stereodiastereoisomers, results can result in compounds with profoundly different pharmacological performances, including potency, toxicity, efficacy, cellular uptake, and biodistribution.

In this regard, a traditional phosphorothioate oligonucleotide, 16 nucleotides in length contains up to 2¹⁵ different diastereoisomers, potentially over 32,000 pharmacologically distinct compounds. There is considerable potential in identifying a pharmacologically optimal compound from such as mixture, a possibility which may result in compounds which have far greater therapeutic potential than the standard stereorandom phosphorothioate.

Previously, workers have focused on the identification of stereodefined motifs, typically contiguous patterns of Sp or Rp stereodefined motifs which can confer for example a more effective RNaseH activity.

For example, WO2015/107425 reports on the chiral designs of chirally defined oligonucleotides, and reports in FIG. 22 that selective positioning of the 3′-SSR-5′ site allows moderate differentiation in RNA cleavage rate but enhanced discrimination between allelic variants for oligonucleotide ONT-453.

In our studies using LNA gapmer oligonucleotides in vitro in cell based assays, as well as in vivo, we have noted a profound unpredictability when applying a pattern of stereodefined phosphorothioate linkages from one LNA gapmer to another, and that in general, it is necessary to optimize the stereochemistry of the backbone linkages of antisense oligonucleotides on an individual basis.

This unpredictability raises a serious demand on the discovery paradigm for stereodefined oligonucleotides. Ideally, one would make every possible stereodefined variant of a parent oligonucleotide, and select the optimized “child” oligonucleotide which has the best pharmacological profile. Whilst possible, such a massively parallel discovery process would be very resource demanding.

WO2016/96938 discloses a method of optimising phosphorothioate oligonucleotides for greater tolerability by the creation of a library of stereodefined variants and selection from the library of variants which have a reduced toxicity. WO'938 includes one aspect where iterative screening allows for further improvement (a serial drug discovery process). The examples of WO'938 include compounds where only a few internucleoside linkages in the compounds are stereodefined, the remainder being stereorandom.

The present inventors have identified that such a “sub-library” approach allows for a more effective drug discovery process, where rather than a screening of 2¹⁵ diastereoisomers of a 16mer, we could screen sub-libraries where some but not all phosphorothioate internucleoside linkages had a predetermined stereodefinition, greatly simplifying the library complexity, and that selecting improved “sub-library” compounds.

WO 2016/079181 discloses numerous fully stereodefined LNA gapmers of sequence Gₛ^{m}CₛaₛaₛgₛcₛaₛtₛcₛcₛtₛGₛT, where capital letters represent beta-D-oxy LNA nucleotides, which were evaluated in an ex-cellular RNase H assay.

Wan et al., Nucleic Acid Research, 2014 Dec. 16; 42(22):13456-68 reports that whilst stereodefined internucleoside linkages may affect ex-cellular RNaseH activity, controlling the chirality of the PS linkage in the gap region of the tested RNase H active gapmer ASOs provided no discernable benefit for therapeutic applications relative to the mixture of stereo-random PS ASOs.

Iwamoto et al., Nature Biotechnology, 21 Aug. 2017; doi:10.1038/nbt.3948 discloses phosphorothioate (PS) stereochemistry substantially affects the pharmacologic properties of ASOs and reports that Sp-configured PS linkages are stabilized relative to Rp, providing stereochemical protection from pharmacologic inactivation of drugs. They also elucidated a triplet stereochemical code in the stereopure ASOs, 3′-SpSpRp, that promotes target RNA cleavage by RNase H1 in vitro and provides a more durable response in mice than stereorandom ASOs. Notably, the supplementary data in Iwamoto et al., indicates that there is a lack of predictability with regards potency from in vitro to in vivo (see suppl FIG. 5).

The upredictability of the pharmacological properties of stereodefined antisense oligonucleotides is further illustrated by the work of the present inventors, who address the problem of unpredictability of stereodefined oligonucelotides by employing sub-libraries where only part of the antisense oligonucleotide has stereodefined phosphorothioate internucleoside linkages, and the remaining part comprises or is stereorandom phosphorothioate linked nucleosides.

### SUMMARY OF INVENTION

The present inventors have found that the sub-library approach allows for preferred stereodefined motifs and their specific position within the oligonucleotide to be determined. In this respect, the sub-library approach reduced the complexity of oligonucleotide libraries and overcomes some of the unpredictability seen with fully stereodefined oligonucleotides.

The sub-library approach allows for the identification of, and optimal position of, short stereodefined motifs which are associated with an improved pharmacologically relevant property, whilst avoiding some of the inherent unpredictability associated with fully stereodefined oligonucleotides.

The present inventors have also discovered that the discovery process for identifying optimised fully stereodefined oligonucleotides can be greatly simplified by combining preferred short stereodefined motifs identified from positionally different sub-libraries into a single compound.

The methods of the present invention therefore provide for the efficient discovery of position dependent stereodefined motifs which can either be used as therapeutic oligonucleotides, or may be used as a less complex starting point for discovering compounds with further stereodefined internucleoside linkages or fully stereodefined compounds.

By generating a series of independent sub-libraries incorporating a short stereodefined motif in an otherwise stereorandom oligonucleotide, where the position of the motif differs between each sub-library, the optimal position for a stereodefined motif can be identified. This is referred to as a motif “walk” approach, where a motif can be sequentially shifted one position in each sub-library. The motif “walk” approach may be performed across and entire compound, or a region thereof, for example within the gap region of a gapmer. The short motif may be a contiguous motif or may be a dis-contiguous motif. In its simplest form, the motif may be as short as a single internucleoside position, Rp, or Sp, in an otherwise fully stereorandom phosphorothioate oligonucleotide. For example a comprehensive library based on a stereorandom 16mer parent oligonucleotide would have 15 “Sp” sub-libraries, each Sp sub-library having a Sp at one of the possible 15 positions, the remaining internucleoside linkage being stereorandom, and 15 “Rp” sub libraries, each Rp sub-library having a Rp at one of the possible 15 positions, the remaining internucleoside linkage being stereorandom. In this respect by screening just 30 oligonucleotide sub-libraries, it is possible to explore the maximum stereochemical diversity in the backbone.

A similar approach may be performed utilising short regions of 2 or more contiguous stereodefined internucleoside linkages. For example 4 duplex linkage motifs, such as RR, SS, SR, RS may be walked through the compound, or 8 triplex linkage motifs RRR, RSR, RRS, RSS, SSS, SRS, SSR, SRR, or the 16 quadruplex linkage motifs, RRRR, RRRS, RRSR; RSRR, RRSS; RSRS; RSSR; RSSS, SSSS, SSSR; SSRS; SRSS; SSRR; SRSR; SRRS, SRRR.

Or for linkage motifs of 5 linkages: RRRRR,RRRRS, RRRSR,RRRSS, RRSRR; RRSRS, RSRRR, RRSSR; RSRSR; RSSRR; RSSSR, SSSSR, SSSRR; SSRSR; SRSSR; SSRRR; SRSRR; SRRSR, SRRRR, RSRRS, RRSSS; RSRSS; RSSRS; RSSSS, SSSSS, SSSRS; SSRSS; SRSSS; SSRRS; SRSRS; SRRSS, or SRRRS.

Using the motif walk approach it is possible to select stereodefined variant “sub-libraries” with a pronounced enhancement in in vitro or in vivo potency. By combining motifs from the selected sub-libraries which illustrate improved properties, further stereodefined compounds, including fully stereodefined compounds can be identified which retain the improvements identified in the selected sub-libraries, or are further improved.

The present inventors have developed a multiple parallel library screening approach where multiple exclusive or over-lapping short sub-regions or motifs of stereodefined phosphorothioate linked nucleosides are optimised to identify enhanced sub-libraries, and stereodefined internucleoside linkage patterns from each of the selected (improved) sub-libraries are then combined to produce an enhanced stereodefined compound.

The invention provides a method for identifying improved stereodefined phosphorothioate variants of an antisense oligonucleotide, said method comprising the steps of:
a. Providing a parent oligonucleotide, with a defined sequence and nucleoside modification pattern;
b. Generating a library of stereodefined phosphorothioate oligonucleotides which retain the defined sequence and nucleoside modification pattern of the parent oligonucleotide, wherein either
   (i) each member of the library is a sub-library comprising a mixture of stereodefined phosphorothioate antisense oligonucleotides diastereoisomers, wherein each member of the mixture comprises a stereodefined internucleoside motif region, wherein, the stereodefined internucleoside motif region is a common region of 2-8, such as 3-8 contiguous nucleosides, wherein the remaining internucleoside linkages comprise stereorandom phosphorothioate internucleoside linkages; wherein, the length and the position of each common stereodefined internucleoside linkage motif region is the same between each member of the library; and wherein, each member of the library comprises a different common stereodefined internucleoside motif in the stereodefined internucleoside motif region;
   or
   (ii) wherein each member of the library is a sub-library comprising a mixture of stereodefined phosphorothioate antisense oligonucleotides diastereoisomers, wherein each member of a mixture comprises a common stereodefined internucleoside linkage motif at the same position in the oligonucleotide, wherein the remaining internucleoside linkages comprise stereorandom phosphorothioate internucleoside linkages; wherein each member of the library comprises the same common stereodefined internucleoside linkage motif, wherein the position of the common stereodefined internucleoside linkage motif differs between each member of the library;
c. Screening each member of the library generated in step b) for at least one improved property, such as improved potency and/or reduced toxicity, as compared to the parent oligonucleotide;
d. Identifying one or more members of the library which have the improved property.

The invention provides a method for identifying improved stereodefined phosphorothioate variants of an antisense oligonucleotide, said method comprising the steps of:
a. Providing a parent oligonucleotide, with a defined sequence and nucleoside modification pattern;
b. Generating a library of stereodefined phosphorothioate oligonucleotides which retain the defined sequence and nucleoside modification pattern of the parent oligonucleotide, wherein each member of the library is a sub-library comprising a mixture of stereodefined phosphorothioate antisense oligonucleotides diastereoisomers, wherein each member of the mixture comprises a stereodefined internucleoside motif region, wherein, the stereodefined internucleoside motif region is a common region of 2-8, such as 3-8 contiguous nucleosides, wherein the remaining internucleoside linkages comprise stereorandom phosphorothioate internucleoside linkages; wherein, the length and the position of each common stereodefined internucleoside linkage motif region is the same between each member of the library; and wherein, each member of the library comprises a different common stereodefined internucleoside motif in the stereodefined internucleoside motif region;
c. Screening each member of the library generated in step b) for at least one improved property, such as improved potency and/or reduced toxicity, as compared to the parent oligonucleotide;
d. Identifying one or more members of the library which have the improved property.

The invention provides a method for identifying improved stereodefined phosphorothioate variants of an antisense oligonucleotide, said method comprising the steps of:
a. Providing a parent oligonucleotide, with a defined sequence and nucleoside modification pattern;
b. Generating a library of stereodefined phosphorothioate oligonucleotides which retain the defined sequence and nucleoside modification pattern of the parent oligonucleotide, wherein each member of the library is a sub-library comprising a mixture of stereodefined phosphorothioate antisense oligonucleotides diastereoisomers, wherein each member of a mixture comprises a common stereodefined internucleoside linkage motif at the same position in the oligonucleotide, wherein the remaining internucleoside linkages comprise stereorandom phosphorothioate internucleoside linkages; wherein each member of the library comprises the same common stereodefined internucleoside linkage motif, wherein the position of the common stereodefined internucleoside linkage motif differs between each member of the library;
c. Screening each member of the library generated in step b) for at least one improved property, such as improved potency and/or reduced toxicity, as compared to the parent oligonucleotide;
d. Identifying one or more members of the library which have the improved property.

The invention provides for a compound (such as an LNA gapmer oligonucleotide) selected from the group consisting of

(SEQ ID NO 1)
5′-G_{srP}^{m}C_{ssP}a_{ssP}a_{srP}g_{srP}c_{ssP}a_{srP}t_{srP}c_{ssP}c_{srP}t_{ssP}G_{ssP}
T-3′
or
(SEQ ID NO 1)
5′-G_{srP}^{m}C_{ssP}a_{srP}a_{srP}g_{srP}c_{ssP}a_{ssP}t_{srP}c_{ssP}c_{srP}t_{ssP}G_{ssP}
T-3′
or
(SEQ ID NO 1)
5′-G_{srP}^{m}C_{ssP}a_{srP}a_{srP}g_{srP}c_{ssP}a_{ssP}t_{srP}c_{srP}c_{ssP}t_{srP}G_{ssP}
T-3′

wherein capital letters represent a beta-D-oxy LNA nucleoside (2′-O—CH2-4′ bridged nucleoside in the beta-D-orientation), lower case letters represent a DNA nucleoside, subscript _{ssP} represents an Sp stereodefined phosphorothioate linkage, and _{srP} represents a Rp stereodefined phosphorothioate linkage. ^{m}C represents a 5-methyl cytosine LNA nucleoside, or a pharmaceutically acceptable salt thereof.

The invention provides for a conjugate comprising the LNA gapmer oligonucleotide according to the invention, and at least one conjugate moiety covalently attached to said oligonucleotide. In some embodiments the conjugate moiety is capable of binding to the asialoglycoprotein receptor, such as a GalNAc conjugate moiety.

In an embodiment of the present invention each member of the library generated in step b) is screened for at least one improved property, such as improved potency and/or reduced toxicity and/or improved selectivity, as compared to the parent oligonucleotide.

The invention provides for a pharmaceutical composition comprising the LNA gapmer oligonucleotide or conjugate according to the invention, and a pharmaceutically acceptable diluent, carrier, salt and/or adjuvant.

The invention provides for a pharmaceutically acceptable salt of the compound, such as the LNA gapmer oligonucleotide, or conjugate according to the invention

The invention provides for the compound, such as the LNA gapmer oligonucleotide or conjugate, according to the invention, for use in medicine.

The invention provides for the compound, such as the LNA gapmer oligonucleotide or conjugate, according to the invention for use in the treatment of cancer.

The invention provides for the use of the compound, such as the LNA gapmer oligonucleotide or conjugate according to the invention for the manufacture of a medicament for treatment of cancer.

### BRIEF DESCRIPTION OF FIGURES

FIG. 1: Sub motif optimization illustration. In this figure we illustrate three parallel optimization methods of a parent LNA gapmer oligonucleotide, one where the library comprises of compounds A1-A16, introducing 16 sub-libraries each with one of the 16 possible unique quadruplex stereodefined motifs in internucleoside linkages 1-4, the second compounds B1-B16, introducing 16 sub-libraries each with one of the 16 possible unique quadruplex stereodefined motifs in internucleoside linkages 5-8, and the third compounds C1-16, introducing 16 sub-libraries each with one of the 16 possible unique quadruplex stereodefined motifs in internucleoside linkages 9-12. Each library is screened for an improved property, and the individual sub-libraries exhibiting the improved property is identified.

FIG. 2: Combinatorial sub library sub motif optimization. In this figure we illustrate that the three libraries represented and screened in FIG. 1 may provide three optimized sub-libraries, one from each of the three libraries. The stereodefined motifs from two or more of the optimized sub-libraries (from separate libraries) may then be combined into a single compound, which may be further assessed for the improved property or different improved properties. The identified optimized compound may be subjected to further optimization method steps, for example to optimize a different property.

FIG. 3: Single Position Oligonucleotide Walk—Stereorandom Background. In this figure we illustrate the motif walk method of the invention, in this case using a single R or S stereodefined internucleoside linkage which is “walked through” in a series of sub-libraries, in an otherwise stereorandom backbone. Such an approach is ideal in identifying internucleoside positions within an oligonucleotide where one of the diastereosisomers (R or S) is associated, positively or negatively, with a pharmacologically property of the oligonucleotide (such as an improved property). It will also identify specific internucleoside positions wherein one of R or S is essential to achieve the desired property (such as potency). This information may be used to prepare a sub-library compound where all the individual diastereoisomers have the beneficial or essential chiral configuration, either as an improved oligonucleotide or as a new parent oligonucleotide for further optimization iterations. Alternatively as illustrated, the information relating to the most beneficial or essential chiral configuration (R or S) at each internucleoside position may be combined into a single optimized compound which may be further assessed for the improved property or different improved properties. The identified optimized compound may be subjected to further optimization method steps, for example to optimize a different property.

FIG. 4: Single Position Oligonucleotide Walk—Stereopure Background. In this figure we illustrate the motif walk method of the invention, in this case using a single R or S stereodefined internucleoside linkage which is “walked through” in a series of sub-libraries, in an otherwise stereopure backbone of the other stereodefined linkage. This may be used to identify essential or preferred stereodefined internucleoside positions and entantiomers (R or S) within the oligonucleotide, and allow for the identification of sub-libraries which may be subjected to further optimization, as described herein.

FIG. 5: Duplex Walk—Stereorandom Background. In this figure we illustrates a duplex walk using the four possible stereodefined duplex motifs, SS, RS, SR and RR.

FIG. 6: Triplex Walk—Stereorandom Background. In this figure we illustrates a triplex walk using the eight possible stereodefined triplex motifs, SSS, SSR, RSS, RSR, SRS, SRR, RRR and RRS.

FIG. 7: Sub-Motif Walk—Stereorandom Background. In this figure we illustrate a sub-motif walk, in this case and RSSR walk, to identify the optimal position for a stereodefined sub-motif within the oligonucleotide.

FIG. 8: In vitro Hif-1alpha mRNA knockdown after incubation of Hela cells for 3 days with fully stereodefined LNA oligonucleotides at 5 μM concentration (via. gymnosis).

FIG. 9a: Hifa1 13mer—Position 1-4 sublibraries

FIG. 9b: Hifa1 13mer—Position 5-8 sublibraries

FIG. 9c: Hifa1 13mer—Position 9-12 sublibraries

FIG. 10: full stereorandom screen figure—highlighting RSSR position 5 as a preferred motif

FIG. 11: full stereorandom screen figure—highlighting RSSR position dependence—RSSR effect not seen at position 6 for the Hif1alpha compound. preferred motif.

FIG. 12: In vivo target knock-down in the liver, illustrating the in vivo potency of the RSSR position 5 compound (#18) vs a position 6 RSSR compound (#21), and the parent compound (#39).

FIG. 13a: In vivo liver content analysis, illustrating the in vivo potency of the RSSR position 5 compound (#18) is associated with an increase is tissue uptake in liver as compared to a position 6 RSSR compound (#21), and the parent compound (#39).

FIG. 13b: In vivo kidney content analysis, illustrating the in vivo potency of the RSSR position 5 compound (#18) is associated with an increase is tissue uptake in kidney as compared to a position 6 RSSR compound (#21), and the parent compound (#39).

FIG. 14a: In vivo target knock down in the liver: Evaluation of the position 5 (#42) vs position 6 RSSR (#41) based motifs in an independent ApoB targeting compound. As with the Hif1alpha position 5 RSSR compound, there was a dramatic increase in in vivo potency as compared to the stereorandom parent compound, illustrating that the position 5 RSSR motif was transferable betwee compounds of different sequence and target. The position 6 RSSR compound (#41) was less potent than the parent compound, again confirming the positional dependence of stereodefined sub-motifs within an antisense compound.

FIG. 14b: In vivo target knock down in the kidney: Evaluation of the position 5 (#42) vs position 6 RSSR (#41) based motifs in an independent ApoB targeting compound. As with the Hif1alpha position 5 RSSR compound, there was a dramatic increase in in vivo potency as compared to the stereorandom parent compound, illustrating that the position 5 RSSR motif was transferable between compounds of different sequence and target. The position 6 RSSR compound (#41) was less potent than the parent compound, again confirming the positional dependence of stereodefined sub-motifs within an antisense compound.

FIG. 15a: In vivo liver content analysis, illustrating the in vivo potency of the RSSR position 5 compound (#42) is associated with an increase is tissue uptake inliver as compared to a position 6 RSSR compound (#41), and the parent compound (#40).

FIG. 15b: In vivo kidney content analysis, illustrating the in vivo potency of the RSSR position 5 compound (#42) is not associated with an increase is tissue uptake in kidney as compared to the parent compound (#40), but kidney uptake is higher than the position 6 compound (#41).

FIG. 16: Reduction in total serum cholesterol from the in vivo experiment comparing ApoB targeting parent compound (#40), and the position 5 RSSR (#42) and position 6 RSSR (#41) compound illustrating a dramatic increase in in vivo pharmacology of the position 5 RSSR compound (#42) as compared to both the parent compound (#40) and the position 6 RSSR compound (#41).

FIG. 17: Statistical analysis of 263 16mer gapmer compounds with a 3-9-4 design, illustrating that for an independent sequence (as compared to the previous examples), and an oligonucleotide of a different length (16) and design, the position 5 RSSR motif was a preferred motif resulting in highly potent compounds.

FIG. 18: Statistical analysis of 263 16mer gapmer compounds with a 3-9-4 design, illustrating that for an independent sequence (as compared to the previous examples), and an oligonucleotide of a different length (16) and design, the position 5 RSSR motif was a preferred motif resulting in highly potent compounds.

FIG. 19: Illustration of the exploitation of property diversity between stereodefined child oligonucleotides identified using the methods of the invention to identify individual diastereoisomers with refined properties.

FIG. 20: Single position motif walk. A stereorandom 19mer LNA gapmer parent compound which was selected, and two libraries were generated, one where a single Sp stereodefined internucleoside linkage was walked across the oligonucleotide, so that each member of the library differed with respect to the position of the Sp stereodefined linkage, and a second library where a single Rp stereodefined internucleoside linkage was walked across the oligonucleotide, so that each member of the library differed with respect to the position of the Rp stereodefined linkage. In this experiment, the remaining internucleoside linkages were stereorandom. Each member of each library was assayed for potency against the mRNA target in U251 cells using gymnotic delivery of 1 μM (See example 6 for the methodology). mRNA target knock-down for each library member was determined. The results identified 4 positions where the stereodefinition (Sp or Rp) was a notable determinant of oligonucleotide potency, and 7 positions where the stereochemistry was not a relevant determinant of oligonucleotide potency. This approach allows the design of partially stereodefined compounds which comprise the preferred stereodefined internucleoside linkage at the stereo-relevant positions, and stereorandom internucleoside linkages at the stereo-irrelevant positions. Such optimized sub-library compounds may be used in further optimization methods (e.g. of the invention), to identify further stereodefined variants, including fully stereodefined variants, which have further improved properties.

FIG. 21: Sub-library approach: a stereorandom 19mer LNA gapmer parent compound was selected, and two 32 sub-libraries were generated. The 19mer LNA gapmer comprises LNAs in the 5′ and the 3′ end as indicated on FIG. 21 with the changes in shadings. Lighter shading indicates DNA nucleosides, while darker shading indicates the position of an LNA nucleoside. The first sub-library was created by stereodefining the five first internucleoside linkages in the 5′ end. The second library was created by stereodefining last five internucleoside linkages in the 3′ end. In this experiment, the remaining internucleoside linkages were stereorandom. On FIG. 21, the arrows indicate where the internucleosides have been stereodefined.

FIG. 22: shows the results of an assay in which each member of the first sub-library of FIG. 21 was assayed for potency against the mRNA target in U251 cells using gymnotic delivery of 1 μM (See example 6 for the methodology). mRNA target knock-down for each library member was determined.

FIG. 23: shows the results of an assay in which each member of the second sub-library of FIG. 21 was assayed for potency against the mRNA target in U251 cells using gymnotic delivery of 1 μM (See example 6 for the methodology). mRNA target knock-down for each library member was determined. The results show that the first library comprises a larger number of potent oligonucleotides with less variability than the second library. This approach allows the design of partially stereodefined compounds which comprise the preferred stereodefined internucleoside linkage at the stereo-relevant positions, and stereorandom internucleoside linkages at the stereo-irrelevant positions. Such optimized sub-library compounds may be used in further optimization methods (e.g. of the invention), to identify further stereodefined variants, including fully stereodefined variants, which have further improved properties.

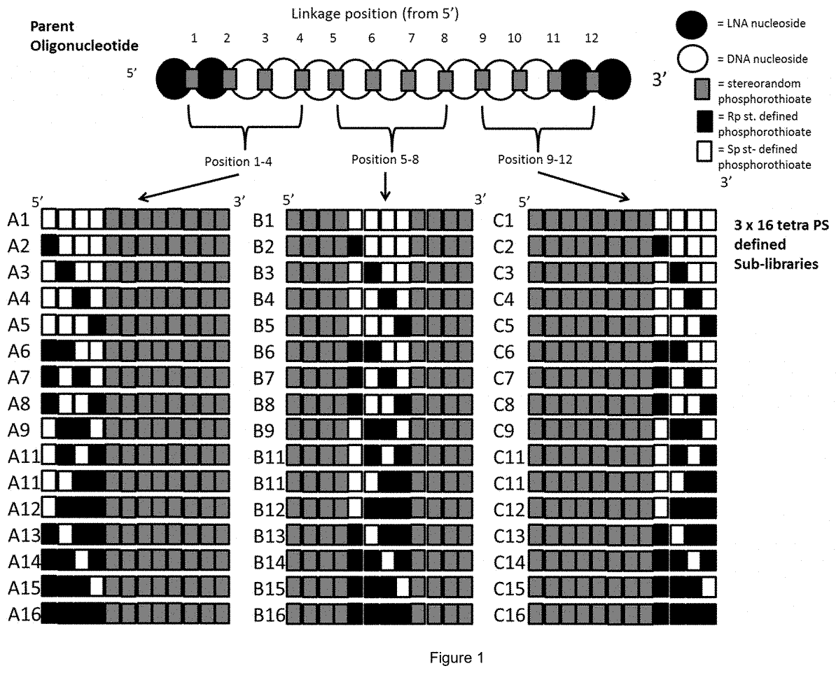

### DETAILED DESCRIPTION OF THE INVENTION

The invention provides methods for identifying improved stereodefined variants of a parent oligonucleotide by employing a library of sub-libraries, which is based upon a “stereodefined motif walk”, where a short stereodefined motif is positioned at different internucleoside positions between each member of the library (Positional Diversity).

The invention provides methods for identifying improved stereodefined variants of a parent oligonucleotide by employing a library of sub-libraries, which is based upon the creation of different stereodefined motifs at the same internucleoside position within the oligonucleotide, where each member of the library has a unique stereodefined motif at the designated position of the oligonucleotide.

The methods of the invention may be used reiteratively and/or in combination, and may further be combined with stereorandom discovery methods.

Stereodefined Motif Walk:

The invention provides for a method for identifying improved stereodefined phosphorothioate variant of an antisense oligonucleotide, said method comprising the steps of:
a. Providing a parent phosphorothioate oligonucleotide, with a defined sequence and nucleoside modification pattern;
b. Generating a library of stereodefined phosphorothioate oligonucleotides which retain the defined sequence and nucleoside modification pattern of the parent oligonucleotide, wherein each member of the library [each member may be referred to as a sub-library of diastereosisomers] comprises a mixture of stereodefined phosphorothioate antisense oligonucleotides, wherein each member of a mixture [sub-library] comprises a common stereodefined internucleoside motif at the same position in the oligonucleotide, wherein the remaining internucleoside linkages comprise stereorandom phosphorothioate internucleoside linkages; wherein each member of the library comprises the same common stereodfined internucleoside linkage motif, wherein the position of the common stereodefined internucleoside linkage motif differs between each member of the library;
c. Screening each member of the library generated in step b) for at least one improved property, such as improved potency and/or reduced toxicity, as compared to the parent oligonucleotide.
d. Identifying one or more members of the library which have the improved property.

In some embodiments, in designing the library the common stereodefined internucleoside motif is shifted by 1 internucleoside position between members of the library such that the common stereodefined internucleoside motif is “walked” across the internucleoside linkage backbone of the oligonucleotide. It will be understood that such a motif-walk approach may be applied across the entire internucleoside linkage backbone of the oligonucleotide, or contiguous nucleotide sequence thereof, or in some embodiment, part of the oligonucleotide, or contiguous nucleotide sequence thereof (e.g. across the gap region of a gapmer).

In some embodiments of the method of the invention, such as the motif walk method, the length of the common stereodefined internucleoside linkage motif is 1-6 internucleoside linkages, such as 2, 3, 4 or 5 internucleoside linkages.

In some embodiments of the method of the invention, such as the motif walk method, the common stereodefined internucleoside linkage motif comprises is either:
a duplex linkage motif selected from the group consisting of SS; RR; RS and SR; or
a triplex linkage motif selected from the group consisting of RRR, RSR, RRS, RSS, SSS, SRS, SSR, and SRR; or
a quadruplex linkage motif selected from the group consisting of RRRR, RRRS, RRSR; RSRR, RRSS; RSRS; RSSR; RSSS, SSSS, SSSR; SSRS; SRSS; SSRR; SRSR; SRRS, and SRRR; or
a pentaplex linkage motif selected from the group consisting of RRRRR,RRRRS, RRRSR,RRRSS, RRSRR; RRSRS, RSRRR, RRSSR; RSRSR; RSSRR; RSSSR, SSSSR, SSSRR; SSRSR; SRSSR; SSRRR; SRSRR; SRRSR, SRRRR, RSRRS, RRSSS; RSRSS; RSSRS; RSSSS, SSSSS, SSSRS; SSRSS; SRSSS; SSRRS; SRSRS; SRRSS, and SRRRS.

In some embodiments of the method of the invention, such as the motif walk method, according the common stereodefined internucleoside linkage motif is or comprises RSSR. The present inventors have found that in some instances the RSSR motif may confer enhanced properties to an oligonucleotide, but that this is highly position dependent within an oligonucleotide, and that shifting an RSSR position by a single internucleoside position can completely remove any benefit associated with the RSSR motif.

In some embodiments, the remaining internucleoside linkages (background backbone linkages), other than the stereodefined internucleoside motif e.g used in the motif walk method, are stereorandom internucleoside linkages, such as stereorandom phosphorothioate internucleoside linkages. In some embodiments, the background backbone linkages are stereopure linkages, i.e. are all R or are all S (such as all Rp or all Sp) stereodefined linkages. In some embodiments, the backbone linkages may comprise one or more stereodefined internucleoside linkage, such as a linkage which has been previously identified as being beneficial such as being associated with an improved property.

In some embodiments of the method of the invention, the library is a comprehensive oligonucleotide walk, i.e. the library comprises all positional variants of the common stereodefined internucleoside linkage motif within the oligonucleotide, the contiguous nucleotide sequence thereof, or gapmer region F, G or F′, or combined sequence F-G-F′.

In some embodiments, two sub-libraries are created by stereodefining internucleoside linkages in the 5′ end or the 3′ end region of a gapmer. In an embodiment, for example 1, 2, 3, 4 or 5 consecutive internucleoside linkages are stereodefined at the 5′ end. In an embodiment, 1, 2, 3, 4 or 5 consecutive internucleoside linkages are stereodefined at the 3′ end, while the rest of the internucleoside linkages are stereorandom. Such stereodefinition can be selected among pentaplex linkage motifs as described herein.

In some embodiments of the method of the invention, the improved property is selected from the group consisting of in enhanced activity, enhanced potency, enhanced efficacy, enhanced specific activity, reduced toxicity, such as reduced hepatotoxicity or reduced nephrotoxicity, altered biodistribution, enhanced cellular or tissue uptake, and/or enhanced target specificity.

In some embodiments of the method of the invention, the improved property is assayed in vitro.

In some embodiments, the antisense oligonucleotides is an RNase H recruiting oligonucleotides such as antisense oligonucleotide gapmer oligonucleotides.

In some embodiments, the antisense oligonucleotides are LNA gapmer oligonucleotides. In some embodiments, the length of the antisense oligonucleotide is 7-26 nucleotides in length, such as 12-24 nucleotides in length.

Contiguous Sub-Motif Optimization

The invention provides for a method for identifying improved stereodefined phosphorothioate variant of an antisense oligonucleotide, said method comprising the steps of:
a. Providing a parent oligonucleotide, with a defined sequence and nucleoside modification pattern;
b. Generating a library of stereodefined phosphorothioate oligonucleotides which retain the defined sequence and nucleoside modification pattern of the parent oligonucleotide,
   wherein, each member of the library is a sub-library comprising a mixture of stereodefined phosphorothioate antisense oligonucleotides enantiomer, wherein each member of the mixture [sub-library] comprises a stereodefined internucleoside motif region,
   wherein, the stereodefined internucleoside motif region is a common region of 2-8, such as 3-8, such as 4-8 contiguous nucleosides, wherein the remaining internucleoside linkages comprise stereorandom phosphorothioate internucleoside linkages;
   wherein, the length and the position of each stereodefined internucleoside linkage motif region is the same between each member of the library;
   and wherein, each member of the library comprises a different common stereodefined internucleoside motif in the stereodefined internucleoside motif region;
c. Screening each member of the library generated in step b) for at least one improved property, such as improved potency and/or reduced toxicity, as compared to the parent oligonucleotide;
d. Identifying one or more members of the library which have the improved property.

The above method of the invention relates to the optimization of a defined sub-region of the backbone internucleoside linkages by the creation of a library of variant oligonucleotides which each have a different stereodefined sub-motif within the sub-region. This approach allows for the selection of stereodefined variants which have an optimized stereodefined sub-motif across the sub-region. By way of example, the library comprises members where each member has a unique internucleoside motif positioned at the same position between each member, e.g. for a for a dinucleotide, it will result in two variants (R or S); for a trinucleotide region, this will result in four variants (library members), with either a RR, SS, SR, or RS internucleoside motif. For a tetranucleotide region, this will result in eight variants (library members) with either a RRR, RSR, RRS, RSS, SSS, SRS, SSR, or SRR internucleoside motif.

For a 5 nucleotide region, it will result in 16 variants (library members), with either RRRR, RRRS, RRSR; RSRR, RRSS; RSRS; RSSR; RSSS, SSSS, SSSR; SSRS; SRSS; SSRR; SRSR; SRRS, or SRRR.

For a 6 nucleotide region, it will result in 32 variants (library members), with either RRRRR,RRRRS, RRRSR,RRRSS, RRSRR; RRSRS, RSRRR, RRSSR; RSRSR; RSSRR; RSSSR, SSSSR, SSSRR; SSRSR; SRSSR; SSRRR; SRSRR; SRRSR, SRRRR, RSRRS, RRSSS; RSRSS; RSSRS; RSSSS, SSSSS, SSSRS; SSRSS; SRSSS; SSRRS; SRSRS; SRRSS, or SRRRS.

Suitably, in some embodiments, the remaining internucleoside linkages are stereorandom internucleoside linkages, or the remaining phosphorothioate internucleoside are stereorandom phosphorothioate internucleoside linkages. It is recognized however that in some embodiments one or more of the remaining internucleoside linkages in the members of the libraries may also be stereodefined, e.g. the one or more, or all of the remaining internucleoside linkages may be the result of the optimization of the stereodefined internucleoside linkages elsewhere in the oligonucleotide or contiguous nucleotide sequence, in which case each member will retain such optimized stereodefined internucleoside linkages.

Alternatively, in some embodiments, the stereodefined motif may be a discontinuous motif, comprising the common region of 2-8 such as 3-8 contiguous nucleosides, and further internucleoside linkages positioned elsewhere within the oligonucleotide.

In some embodiments of the method of the invention, such as the contiguous motif optimization method, the length of each stereodefined internucleoside linkage motif region is 3, 4, 5 or 6 contiguous nucleotides (or 2, 3, 4 or 5 nucleoside linkages), preferably at least 4 contiguous nucleotides (i.e. at least three nucleoside linkages).

In some embodiments of the method of the invention, such as the contiguous motif optimization method, the each stereodefined internucleoside linkage motif region is 3 or 4 nucleosides linkages.

In some embodiments of the method of the invention, such as the contiguous motif optimization method, the library comprises members of each of the possible stereodefined internucleoside linkage motifs within the stereodefined internucleoside linkage motif region.

In some embodiments of the method of the invention, such as the contiguous motif optimization method, each member of the library each comprises
a triplex linkage motif selected from the group consisting of RRR, RSR, RRS, RSS, SSS, SRS, SSR, SRR, or
a quadruplex linkage motif selected from the group consisting of RRRR, RRRS, RRSR; RSRR, RRSS; RSRS; RSSR; RSSS, SSSS, SSSR; SSRS; SRSS; SSRR; SRSR; SRRS, SRRR, or
a pentaplex linkage motif selected from the group consisting of RRRRR,RRRRS, RRRSR,RRRSS, RRSRR; RRSRS, RSRRR, RRSSR; RSRSR; RSSRR; RSSSR, SSSSR, SSSRR; SSRSR; SRSSR; SSRRR; SRSRR; SRRSR, SRRRR, RSRRS, RRSSS; RSRSS; RSSRS; RSSSS, SSSSS, SSSRS; SSRSS; SRSSS; SSRRS; SRSRS; SRRSS, or SRRRS

In some embodiments of the method of the invention, such as the contiguous motif optimization method, the library is comprehensive, i.e. comprises at least one member of each of the possible stereodefined internucleoside motifs of the stereodefined internucleoside motif region, for example the triplex, quadruplex or pentaplex linkage motifs referred to herein.

In some embodiments, the library comprises at least one member of each of the possible duplex stereodefined internucleoside motifs, such as the duplex linkage motifs RR, SS, RS; & SR.

In some embodiments, the library comprises at least one member of each of the possible triplex stereodefined internucleoside motifs, such as the triplex linkage motifs RRR, RSR, RRS, RSS, SSS, SRS, SSR, & SRR.

In some embodiments, the library comprises at least one member of each of the possible quadruplex stereodefined internucleoside motifs, such as the quadruplex linkage motifs RRRR, RRRS, RRSR; RSRR, RRSS; RSRS; RSSR; RSSS, SSSS, SSSR; SSRS; SRSS; SSRR; SRSR; SRRS, & SRRR.

In some embodiments, the library comprises at least one member of each of the possible pentaplex stereodefined internucleoside motifs, such as the pentaplex linkage motifs RRRRR,RRRRS, RRRSR,RRRSS, RRSRR; RRSRS, RSRRR, RRSSR; RSRSR; RSSRR; RSSSR, SSSSR, SSSRR; SSRSR; SRSSR; SSRRR; SRSRR; SRRSR, SRRRR, RSRRS, RRSSS; RSRSS; RSSRS; RSSSS, SSSSS, SSSRS; SSRSS; SRSSS; SSRRS; SRSRS; SRRSS, & SRRRS.

In some embodiments of the method of the invention, such as the contiguous motif optimization method, at least 30%, such as at least 40% or at least 50%, or a majority of, or all the remaining internucleoside linkages within the antisense oligonucleotide of each library member [or sub-library] are stereorandom phosphorothioate internucleoside linkages.

In some embodiments of the method of the invention, such as the contiguous motif optimization method, the method further comprises the steps of
e) Selecting at least one improved oligonucleotide variant identified in step d)
f) Generating a library of stereodefined phosphorothioate oligonucleotides which retain the defined sequence and nucleoside modification pattern and the same stereodefined internucleoside motif of the improved oligonucleotide variant, wherein each member of the library comprises one or more further stereodefined phosphorothioate internucleoside linkages [i.e. not within the stereodefined internucleoside motif or common region], and wherein each member of the library differs with respect to the pattern of further stereodefined phosphorothioate internucleoside linkages,
g. Screening each member of the library generated in step f) for at least one improved property, which may be the same of different improved properties(s) as assayed in step c).

In some embodiments of the method of the invention, such as the contiguous motif optimization method, step b of the method comprises the generation of multiple libraries wherein each library is as defined as in step b and wherein the position of each common stereodefined internucleoside linkage motif region is different between each of the multiple libraries, wherein each library may be a library as defined in any one of the proceeding claims.

In some embodiments of the method of the invention, such as the contiguous motif optimization method, the method further comprises the step of identifying at an improved stereodefined variants from each of the multiple libraries, and preparing a further stereodefined variant which comprises the stereodefined internucleoside linkage motifs of each of the identified improved stereodefined variants from of the multiple libraries.

In some embodiments of the method of the invention, such as the contiguous motif optimization method, at least two or at least three multiple libraries are screened to identify an improved stereodefined variants from each of the multiple libraries, wherein each library is as defined as in step b.

In some embodiments of the method of the invention, such as the contiguous motif optimization method, the further stereodefined variant oligonucleotide or contiguous nucleotide sequence thereof is a fully stereodefined phosphorothioate sequence.

The invention further provides for an improved LNA gapmer phosphorothioate oligonucleotide, wherein the LNA gapmer comprises 5 contiguous nucleosides wherein the pattern of phosphorothioate internucleoside linkages between the 5 contiguous nucleosides is RSSR, wherein R is a Rp stereodefined phosphorothioate internucleoside linkage, and S is an Sp stereodefined phosphorothioate internucleoside linkage, wherein the LNA gapmer has an improved in vitro or in vivo potency as compared to an identical LNA gapmer which has stereorandom phosphorothioate internucleoside linkages. In some embodiments, the RSSR motif is present within the gap region of the gapmer, such as is positioned within the 3′ most nucleoside of region F and the 5′ most nucleoside of region F′.

In some embodiments of the method of the invention, the library is a comprehensive oligonucleotide walk, i.e. the library comprises all positional variants of the common stereodefined internucleoside linkage motif within the oligonucleotide, the contiguous nucleotide sequence thereof, or gapmer region F, G or F′, or combined sequence F-G-F′.

In some embodiments of the method of the invention, the improved property is selected from the group consisting of in enhanced activity, enhanced potency, enhanced efficacy, enhanced specific activity, reduced toxicity, altered biodistribution, enhanced cellular or tissue uptake, and/or enhanced target specificity.

In some embodiments of the method of the invention, the improved property is assayed in vitro.

In some embodiments, the antisense oligonucleotides is an RNase H recruiting oligonucleotides such as antisense oligonucleotide gapmer oligonucleotides.

In some embodiments, the antisense oligonucleotides are LNA gapmer oligonucleotides.

In some embodiments, the length of the antisense oligonucleotide is 7-26 nucleotides in length, such as 12-24 nucleotides in length.

Re-Iterative Screening Method

The invention provides for a method for identifying one or more improved stereodefined phosphorothioate variant of an antisense oligonucleotide, said method comprising the steps of:
a. Providing a parent oligonucleotide, with a defined sequence and nucleoside modification pattern;
b. Generating a library of stereodefined phosphorothioate oligonucleotides which retain the defined sequence and nucleoside modification pattern of the parent oligonucleotide, wherein, each member of the library is a sub-library comprising a mixture of stereodefined phosphorothioate antisense oligonucleotides, wherein each member of the mixture [sub-library] comprises a common stereodefined internucleoside motif, wherein, the common stereodefined internucleoside motif is a common region of 3-8 contiguous nucleosides, wherein the remaining internucleoside linkages comprise stereorandom phosphorothioate internucleoside linkages; wherein, the length and the position of each common stereodefined internucleoside linkage motif is the same between each member of the library; and wherein, each member of the library comprises a different common stereodefined internucleoside motif;
c. Screening each member of the library generated in step b) for at least one improved property, such as improved potency and/or reduced toxicity, as compared to the parent oligonucleotide;
d. Identifying one or more members of the library which have the improved property.
e. Selecting at least one improved member of the library identified in step d)
f. Generating a library of stereodefined phosphorothioate oligonucleotides which retain the defined sequence and nucleoside modification pattern and the same stereodefined internucleoside motif, wherein each member of the library comprises one or more further stereodefined phosphorothioate internucleoside linkages [not within the stereodefined internucleoside motif or common region], and wherein each member of the library differs with respect to the pattern of further stereodefined phosphorothioate internucleoside linkages,
g. Screening each member of the library generated in step f) for at least one improved property, which may be the same of different improved properties(s) as assayed in step c).

Combined Sub-Library Approach

Both the Stereodefined Motif Walk and the Contiguous Sub-Motif Optimization methods of the invention allow for the identification of sub-libraries which have improved properties and which have a reduce complexity (number of distinct diastereoisomers) as compared to a stereorandom parent oligonucleotide.

These approach to identified optimized partially stereodefined (sub-library) compounds may be used iteratively or in combination to further reduce the complexity (number of distinct diastereoisomers) and to further improve the selected compounds. In this respect, either the stereodefined walk to the contiguous sub-motif optimization may identify preferred stereodefined sub-motifs, and that in further rounds of optimization, the preferred stereodefined sub-motifs obtained from either method, may be combined to produce further optimized compounds.

By way of example, by creating several separate libraries of a parent oligonucleotide, where the position of the contiguous sub-motif differs between each library, the present inventors have shown that by combining the identified optimized stereodefined motifs from each library, further enhanced stereodefined oligonucleotides may be identified. Indeed, as illustrated in the examples, the present inventors took a 13mer LNA gapmer stereorandom parent compound, and created three independent libraries, one with a 4 linkage motif in positions 1-4 in an otherwise stereorandom backbone (16 possible variants), the second in positions 5-8 (16 possible variants), and the third in positions 9-12 (16 possible variants)—i.e. a total of 48 compounds. From each of the three libraries the most potent variant was selected, and then the three stereodefined motifs from the three selected compounds was combined into an individual fully stereodefined compound. The resultant fully stereodefined compound was found to have further improved potency, and was identical to a compound which had previously been identified by the screening of a highly complex fully randomized library of fully stereodefined compounds.

The invention provides for a method for identifying improved stereodefined phosphorothioate variant of an antisense oligonucleotide, said method comprising the steps of:
a. Providing a parent oligonucleotide, or a parent oligonucleotide design, with a defined sequence and nucleoside modification pattern;
b. Performing multiple Stereodefined Motif Walk or the Contiguous Sub-Motif Optimization methods of the invention to identify more than one partially stereodefined variants which each have at least one improved property, as compared to the parent oligonucleotide, wherein each more than one identified partially stereodefined variants differ with respect to the position of their stereodefined sub-motif;
c. Prepare a stereodefined variant which comprises the stereodefined sub-motif of the more than one partially stereodefined variants from step b.

In an optional step d., the stereodefined variant prepared in step c., may further be assessed to determine one or more further improved properties which may be the same of different property or properties as those assessed in step b.

It will be recognized that the product of step c. will have a reduce complexity (fewer diastereoisomers) as to the partially stereodefined variants of step b., and may in some embodiments the product of step c. may be a fully stereodefined oligonucleotide (or the contiguous nucleotide sequence thereof may be fully stereoedefined).

In some embodiments, step b comprises multiple contiguous sub-motif optimization steps which may be performed in parallel (at the same time) or in series (sequentially). As illustrated in the examples, in some embodiments, the sub-motifs from each of the Contiguous Sub-Motif Optimization libraries together cover all the phosphorothioate internucleoside linkages of the oligonucleotide, or contiguous nucleotide sequence thereof. This allows for the preparation of a fully stereodefined variant in step c.

### Definitions

Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides.

Antisense Oligonucleotides

The term “Antisense oligonucleotide” as used herein is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs or shRNAs. In some embodiments the antisense oligonucleotides are capable of recruiting RNaseH, such as gapmer oligonucleotides.

Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleotides of the oligonucleotide constitute the contiguous nucleotide sequence. In some embodiments the oligonucleotide comprises the contiguous nucleotide sequence and may optionally comprise further nucleotide(s), for example a nucleotide linker region which may be used to attach a functional group to the contiguous nucleotide sequence. The nucleotide linker region may or may not be complementary to the target nucleic acid.

Nucleotides

Nucleotides are the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In a preferred embodiment the modified nucleoside comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

Stereorandom Phosphorothioate Linkages

Phosphorothioate linkages are internucleoside phosphate linkages where one of the non-bridging oxygens has been substituted with a sulfur. The substitution of one of the non-bridging oxygens with a sulfur introduces a chiral center, and as such within a single phosphorothioate oligonucleotide, each phosphorothioate internucleoside linkage will be either in the S (Sp) or R (Rp) stereoisoforms. Such internucleoside linkages are referred to as “chiral internucleoside linkages”. By comparison, phosphodiester internucleoside linkages are non-chiral as they have two non-terminal oxygen atoms.

The designation of the chirality of a stereocenter is determined by standard Cahn-Ingold-Prelog rules (CIP priority rules) first published in Cahn, R. S.; Ingold, C. K.; Prelog, V. (1966). “Specification of Molecular Chirality”. Angewandte Chemie International Edition. 5 (4): 385-415. doi:10.1002/anie.196603851.

During standard oligonucleotide synthesis the stereoselectivity of the coupling and the following sulfurization is not controlled. For this reason the stereochemistry of each phosphorothioate internucleoside linkages is randomly Sp or Rp, and as such a phosphorothioate oligonucleotide produced by traditional oligonucleotide synthesis actually can exist in as many as 2^{X} different phosphorothioate diastereoisomers, where X is the number of phosphorothioate internucleoside linkages. Such oligonucleotides are referred to as stereorandom phosphorothioate oligonucleotides herein, and do not contain any stereodefined internucleoside linkages. Stereorandom phosphorothioate oligonucleotides are therefore mixtures of individual diastereoisomers originating from the non-stereodefined synthesis. In this context the mixture is defined as up to 2^{X} different phosphorothioate diastereoisomers.

Stereodefined Internucleoside Linkages

A stereodefined internucleoside linkage is an internucleoside linkage which introduces a chiral center into the oligonucleotide, which exists in predominantly one stereoisomeric form, either R or S within a population of individual oligonucleotide molecules.

It should be recognized that stereoselective oligonucleotide synthesis methods used in the art typically provide at least about 90% or at least about 95% stereoselectivity at each internucleoside linkage stereocenter, and as such up to about 10%, such as about 5% of oligonucleotide molecules may have the alternative stereo isomeric form.

In some embodiments the stereoselectivity of each stereodefined phosphorothioate stereocenter is at least about 90%. In some embodiments the stereoselectivity of each stereodefined phosphorothioate stereocenter is at least about 95%.

Stereodefined Phosphorothioate Linkages

Stereodefined phosphorothioate linkages are phosphorothioate linkages which have been chemically synthesized in either the Rp or Sp configuration within a population of individual oligonucleotide molecules, such as at least about 90% or at least about 95% stereoselectivity at each stereocenter (either Rp or Sp), and as such up to about 10%, such as about 5% of oligonucleotide molecules may have the alternative stereo isomeric form.

The stereo configurations of the phosphorothioate internucleoside linkages are presented below

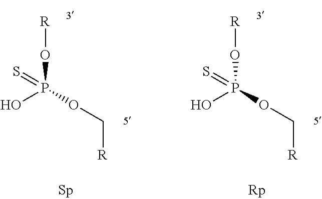

Where the 3′ R group represents the 3′ position of the adjacent nucleoside (a 5′ nucleoside), and the 5′ R group represents the 5′ position of the adjacent nucleoside (a 3′ nucleoside).

Rp internucleoside linkages may also be represented as srP, and Sp internucleoside linkages may be represented as ssP herein.

In some embodiments the stereoselectivity of each stereodefined phosphorothioate stereocenter is at least about 97%. In some embodiments the stereoselectivity of each stereodefined phosphorothioate stereocenter is at least about 98%. In some embodiments the stereoselectivity of each stereodefined phosphorothioate stereocenter is at least about 99%.

In some embodiments a stereoselective internucleoside linkage is in the same stereoisomeric form in at least 97%, such as at least 98%, such as at least 99%, or (essentially) all of the oligonucleotide molecules present in a population of the oligonucleotide molecule.

Stereoselectivity can be measured in a model system only having an achiral backbone (i.e. phosphodiesters) it is possible to measure the stereoselectivity of each monomer by e.g. coupling a stereodefined monomer to the following model-system “5′ t-po-t-po-t-po 3”. The result of this will then give: 5′ DMTr-t-srp-t-po-t-po-t-po 3′ or 5′ DMTr-t-ssp-t-po-t-po-t-po 3′ which can be separated using HPLC. The stereoselectivity is determined by integrating the UV signal from the two possible compounds and giving a ratio of these e.g. 98:2, 99:1 or >99:1.

It will be understood that the stereo % purity of a specific single diastereoisomer (a single stereodefined oligonucleotide molecule) will be a function of the coupling selectivity for the defined stereocenter at each internucleoside position, and the number of stereodefined internucleoside linkages to be introduced. By way of example, if the coupling selectivity at each position is 97%, the resulting purity of the stereodefined oligonucleotide with 15 stereodefined internucleoside linkages will be 0.97¹⁵, i.e. 63% of the desired diastereoisomer as compared to 37% of the other diastereoisomers. The purity of the defined diastereoisomer may after synthesis be improved by purification, for example by HPLC, such as ion exchange chromatography or reverse phase chromatography.

In some embodiments, a stereodefined oligonucleotide refers to a population of an oligonucleotide wherein at least about 40%, such as at least about 50% of the population is of the desired diastereoisomer.

Alternatively stated, in some embodiments, a stereodefined oligonucleotide refers to a population of oligonucleotides wherein at least about 40%, such as at least about 50%, of the population consists of the desired (specific) stereodefined internucleoside linkage motif (also termed stereodefined motif).

For stereodefined oligonucleotides which comprise both stereorandom and stereodefined internucleoside stereocenters, the purity of the stereodefined oligonucleotide is determined with reference to the % of the population of the oligonucleotide which retains the defined stereodefined internucleoside linkage motif(s), the stereorandom linkages are disregarded in the calculation.

Stereodefined Oligonucleotide

A stereodefined oligonucleotide is an oligonucleotide wherein at least one of the internucleoside linkages is a stereodefined internucleoside linkage.

A stereodefined phosphorothioate oligonucleotide is an oligonucleotide wherein at least one of the internucleoside linkages is a stereodefined phosphorothioate internucleoside linkage.

Sub-Library of Stereodefined Oligonucleotides

An oligonucleotide which comprises both stereorandom and stereodefined internucleoside linkages is referred to herein as a sub-library. Sub-libraries are less complex mixtures of the diastereoisomeric mixture of a fully stereorandom oligonucleotide thus representing a sub-set of all possible diastereoisomers. For example, theoretically, a fully phosphorothioate stereorandom 16mer is a mixture of 2¹⁵ diastereoisomer (32768), whereas a sub-library where one of the phosphorothioate internucleoside linkages is stereodefined will have half the library complexity (16384 diastereoisomer), (2 stereodefined linkages=8192 diastereoisomer; 3 stereodefined linkages=4096 diastereoisomer, 4 stereodefined linkages=2048 diastereoisomer, 5 stereodefined linkages=1024 diastereoisomer). (assuming 100% stereoselective coupling efficacy).

A Stereodefined Internucleoside Motif

A stereodefined internucleoside motif, also termed stereodefined motif herein, refers to the pattern of stereodefined R and S internucleoside linkages in a stereodefined oligonucleotide, and is written 5′-3′. For example, the stereodefined oligonucleotide

(SEQ ID NO 1)
5′-G_{srP} C_{ssP} a_{ssP} a_{srP} g_{srP} C_{ssP} a_{srP} t_{srP} C_{ssP}
C_{srP} t_{ssP} G_{ssP} T -3′,

has a stereodefined internucleoside motif of RSSRSRRSRSS.

With respect to sub-libraries of stereodefined oligonucleotides, these will contain a common stereodefined internucleoside motif in an otherwise stereorandom background (optionally with one or more non chiral internucleoside linkages, e.g. phosphodiester linkages).

For example, the oligonucleotide

(SEQ ID NO 1)
5′-Gₛ Cₛ aₛ aₛ g_{srP} c_{ssP} a_{ssP} t_{srP} cₛ cₛ tₛ
Gₛ T -3′

has a stereodefined internucleoside motif of XXXXRSSRXXXX, with X representing a stereorandom phosphorothioate internucleoside linkage (shown as subscript s in the compound). It will be noted that in this example the first 5′ stereodefined internucleoside linkage is the 5^{th} internucleoside linkage from the 5′ end (between the nucleosides at position 4 and 5), and as such the above motif is also referred to as a “RSSR” motif at (internucleoside linkage) position 5.

When the stereodefined internucleoside motif (stereodefined motif) is made up on a series of adjacent stereodefined internucleoside linkages (i.e. positioned between contiguous nucleosides), it is referred to herein as a contiguous stereodefined internucleoside motif (a contiguous stereodefined motif). It will be understood that a contiguous stereodefined motif must comprise two or more adjacent stereodefined internucleoside linkages.

In a sub-library mixture, a stereodefined internucleoside motif may also be dis-contiguous, the stereodefined internucleoside linkages are dispersed with one or more stereorandom internucleoside linkages.

For example the compound

(SEQ ID NO 1)
5′-Gₛ ^{m}C_{ssP} aₛ aₛ g_{srP} C_{ssP} aₛ tₛ Cₛ C_{ssP} t_{srP}
G_{ssP} T -3′

has a dis-contiguous motif XSXXRSXXXSRS

Fully Stereodefined Oligonucleotides

A fully stereodefined oligonucleotide is an oligonucleotide wherein all the chiral internucleoside linkages present within the oligonucleotide are stereodefined. A fully stereodefined phosphorothioate oligonucleotide is an oligonucleotides wherein all the chiral internucleoside linkages present within the oligonucleotide are stereodefined phosphorothioate internucleoside linkages.

It will be understood that, in some embodiments, a fully stereodefined oligonucleotide may comprise one or more, non-chiral internucleosides, such as phosphodiester internucleoside linkages, for example phosphodiester linkages can be used within the flanking regions of gapmers, and/or when linking terminal nucleosides, such as between short regions of DNA nucleosides (biocleavable linker) linking a gapmer sequence and a conjugate group.

In some embodiments of fully stereodefined oligonucleotide, all of the internucleoside linkages present in the oligonucleotide, or contiguous nucleotide region thereof, such as an F-G-F′ gapmer, are stereodefined internucleoside linkages, such as stereodefined phosphorothioate internucleoside linkages.

A Parent Oligonucleotide

A parent oligonucleotide is an oligonucleotide which has a defined nucleobase sequence (motif sequence) and nucleoside modification pattern (design). In the methods of the invention, a parent oligonucleotide is typically an oligonucleotide which is to be improved by the use of the method of the invention by creating one or more libraries where the stereochemistry of one, or more (2+), of the internucleoside linkages is stereodefined and is different to that of the parent oligonucleotide.

In some embodiments, the parent oligonucleotide is a stereorandom phosphorothioate oligonucleotide. In some embodiments, the parent oligonucleotide, or contiguous nucleotide sequence thereof, is a stereorandom phosphorothioate oligonucleotide gapmer. Gapmer oligonucleotides may be useful in inhibiting target mRNA or pre-mRNA expression.

In some embodiments, the parent oligonucleotide, or contiguous nucleotide sequence thereof, is a totalmer or a mixmer. Totalmer and mixmers may be useful in splice switching/modulating oligonucleotides or inhibiting microRNAs for example.

In some embodiments, the parent oligonucleotide may be a sub-library which comprises a common stereodefined motif. The parent oligonucleotide may therefore be a partially stereodefined oligonucleotide, such as a oligonucleotide identified from a previous optimization method.

It will be understood that in some embodiments, it is not necessary to compare the child oligonucleotides for an improved property during the method of the invention, and it is suffice to compare the library members for the improved property. In this regard the parent oligonucleotide may refer to the design of the parent oligonucleotide (sequence and nucleoside modification pattern) which is retained in the members of the library.

Stereodefined Variants (Child Oligonucleotides)

A stereodefined variant of an oligonucleotide is an oligonucleotide which retain the same sequence and nucleoside modifications as a parent oligonucleotide (i.e. the same sequence and nucleoside modification chemistry and design), but differs with respect to one or more stereodefined internucleoside linkages, such as one or more stereodefined phosphorothioate internucleoside linkages (a stereodefined phopshorothioate variant).

A stereodefined variant may be a sub-library, or may be a fully stereodefined oligonucleotide.

A Library of Stereodefined Phosphorothioate Oligonucleotides

A library of stereodefined oligonucleotides comprises numerous members wherein each member is isolated from one another, i.e. in separate pots, and wherein each member has a common sequence and nucleoside modification pattern, wherein each member differs from the other members by virtue of comprising different stereodefined internucleoside motifs.

Each member of the library of stereodefined oligonucleotides may be considered as independent stereodefined variants of a parent oligonucleotide.

Each member of the library may comprise a sub-library, or in some embodiments, each member of the library may be an independent stereodefined oligonucleotide variant.

Improved Property

In order to identify an oligonucleotide which is suitable for use as a therapeutic, it is necessary to identify the rare molecules which have all of the unique properties required to be safe and effective drugs.

A key advantage of generating stereodefined oligonucleotide variants is the ability to increase the diversity across a sequence motif, and select stereodefined oligonucleotides including sub-libraries of stereodefined oligonucleotides, which have improved medicinal chemical properties as compared to a parent oligonucleotide.

A stereodefined oligonucleotide which exhibits one or more improved property as compared to a parent oligonucleotide, or other stereodefined oligonucleotides, is referred to as an improved phosphorotioate variant. Improvement in one or more property is assessed as compared to the parent oligonucleotide, such as a stereorandom parent oligonucleotide.

In some embodiments, the improved medicinal chemical property (or improved property(s)) is/are selected from one or more of optimized affinity, enhanced potency, enhanced specific activity, enhanced tissue uptake, enhanced cellular uptake, enhanced efficacy, altered biodistributiuon, reduced off-target effects, enhanced mismatch discrimination, reduced toxicity, altered serum protein binding, improved duration of action, and enhanced stability.

In some embodiments, the improved property(s) is/are selected from the group consisting of altered or enhanced affinity, enhanced stability, enhanced potency, enhanced efficacy, enhanced specific activity, reduced toxicity, altered or enhanced biodistribution, enhanced duration of action, altered PK/PD, enhanced cellular or tissue uptake, and/or enhanced target specificity.

It will be understood that whilst it is generally desirable to have more potent and less toxic compounds, the benefit of many of the improved properties will depend on the pharmacological challenge the compound needs to address.

Improved Potency and Improved Efficacy

Improved potency refers to the potency of the oligonucleotide in vitro or in vivo, and is typically determined by comparing the level of target modulation, such as target inhibition at a certain dose as compared to a reference compound (parent oligonucleotide). Improved potency may be determined by performing a dose response experiment to determine the dose of the compound which provides 50% inhibition (may be the IC₅₀ level in vitro, or the EC₅₀ level in vivo).

Enhanced efficacy refers to the maximum modulation of the target achieved irrespective of dose, and may be determined in vitro or in vivo.

Reduced Toxicity

In some embodiments the improved property is reduced toxicity, such as reduced hepatotoxicity or reduced nephrotoxicity. In some embodiments, the reduced toxicity is determined in vivo. In some embodiments the reduced toxicity is determined in vitro.

Suitable in vitro assays for determining the hepatotoxicity of antisense oligonucelotides are provided in WO2017067970 and WO2016/096938, hereby incorporated by reference. See also Sewing et al., PLoS One 11 (2016) e0159431.

In some embodiments the parent oligonucleotide is an oligonucleotide which has been determined to be hepatotoxic, either in vitro or in vivo. The child oligonucleotide(s) identified by the method of the invention have a reduced toxicity as compared to the parent oligonucleotide, for example a reduced hepatotoxicity.

In some embodiments the reduced toxicity is reduced hepatotoxicity. Hepatotoxicity of an oligonucleotide may be assessed in vivo, for example in a mouse. In vivo hepatotoxicity assays are typically based on determination of blood serum markers for liver damage, such as ALT, AST or GGT. Levels of more than three times upper limit of normal are considered to be indicative of in vivo toxicity. In vivo toxicity may be evaluated in mice using, for example, a single 30 mg/kg dose of oligonucleotide, with toxicity evaluation 7 days later (7 day in vivo toxicity assay).

Suitable markers for cellular toxicity include elevated LDH, or a decrease in cellular ATP, and these markers may be used to determine cellular toxicity in vitro, for example using primary cells or cell cultures. For determination of hepatotoxicity, mouse or rat hepatocytes may be used, including primary hepatocytes. Suitable markers for toxicity in hepatocytes include elevated LDH, or a decrease in cellular ATP. Primary primate such as human hepatocytes may be used if available. In mammalian hepatocytes, such as mouse, an elevation of LDH is indicative of toxicity. A reduction of cellular ATP is indicative of toxicity, such as hepatotoxicity.

In some embodiments the reduced toxicity is reduced nephrotoxicity. Suitable in vitro assays for determining nephrotoxicity are disclosed in PCT/EP2017/064770, hereby incorporated by reference. See also Moisan et al., Mol. Ther. Nucleic Acids 17 (2017) 89-105. In some embodiments the nephrotoxicity if determined by using an in vitro cell based assay measuring the levels of epidermal growth factor (EGF) as toxicity biomarker, potentially in combination with other biomarkers like adenosine triphosphate (ATP) and kidney injury molecule-1 (KIM-1). An increase in expression of EGF in the supernatant is associated with enhanced nephrotoxicity. Alternatively or in addition, nephrotoxicity may be assessed in vivo, by the use of kidney damage markers including a rise in blood serum creatinine levels, or elevation of kim-1 (kidney injury marker-1) mRNA and/or protein. Suitably mice or rodents may be used.

Other in vitro toxicity assays which may be used to assess toxicity include caspase assays, immune stimulation assays, and cell viability assays, e.g. MTS assays

Enhanced Target Modulation

In some embodiments the improved property may be the ability of the oligonucleotide to modulate target expression, such as via an improved interaction with the cellular machinery involved in modulating target expression, by way of example, an enhanced RNase H activity, an improved splice modulating activity, or an improved microRNA inhibition.

In some embodiments, the improved property is RNaseH specificity, RNaseH allelic discrimination and/or RNaseH activity. In some embodiments, the improved property is other than RNaseH specificity, RNaseH allelic discrimination and/or RNaseH activity. In some embodiments the improved property is improved intracellular uptake.

RNase H Recruitment

Many antisense oligonucleotides operate via RNaseH mediated degredation of the target nucleic acid, and there are numerous reports that RNaseH1 activity may be effected by the stereochemistry of the internucleoside linkages between DNA nucleosides. RNase H activity may be determined in an ex-vivo enzymatic assay, or in an in vitro cell based assay measuring target inhibition. It should be noted that the readout from a cell based assay will incorporate further variables, such as cellular uptake, compartmentalization, and target engagement, as well as an oligonucleotides ability to recruit RNaseH. In some embodiments the improvement in RNaseH activity is accompanied or is characterized by an improved specificity of RNaseH cleavage.

Specificity and Mismatch Discrimination

In some embodiments, the improved property(s) comprise an improvement in the specificity of the antisense oligonucleotide child. Improved specificity relates to an improved ratio to target modulation, such as inhibition as compared to one or more non-target nucleic acids (or unintended targets, often referred to as off-target sequence. The improved property may for example be an improved activity against a disease causing allelic variant as compared to the non disease causing allele. The improved property may therefore be improved mismatch discrimination or target specificity.

Biodistribution

It is often desirable to have an antisense oligonucleotide which is selectively taken up in a target tissue or cell. The methods of the presentment invention may be used to identify child oligonucleotides which have a higher biodistribution or uptake, or higher activity, in the desired target tissue. This may be assessed in vitro by assessing uptake/potency in vitro in cells derived from from the target tissue, such as primary cells. Alternatively or in addition bidistribution may be determined in vivo, either my determining tissue content or target engangment (e.g. inhibition) or by for example use of radio-labelled oligonucelotides followed by whole body or tissue autoradiography.

Affinity Optimisation Alternated, enhanced or optimized affinity refers to an increase or decrease in binding affinity to the target nucleic acid. For RNaseH/gapmer oligonucleotides there is a relationship between the binding affinity of an oligonucleotide and its potency and as such there is often a need to optimize the binding affinity to maximize the potency of an oligonucleotide for the target nucleic acid (See Pedersen et al, Mol Ther Nucleic Acids. 2014 Feb. 18; 3:e149. doi: 10.1038/mtna.2013.72).

Enhanced Stability Enhanced stability refers to the stability of the oligonucleotide from endo-nucleic acid degradation or exo-nucleic acid degradation. Stability against nuclease degradation is often evaluated by determining the stability of the oligonucleotide in serum, or the stability in against snake venom phosphodiesterase (SVPD).

Background Linkages

In the methods of the invention the child oligonucleotides may comprise the stereodefined internucleoside linkage motif in an otherwise stereorandom background, i.e. the remaining internucleoside linkages, or remaining phosphorothioate internucleoside linkages are stereorandom linkages (they have a stereorandom background). However, in some embodiments the child oligonucleotides may comprise one or more further internucleoside linkages which are stereodefined. For motif optimization methods, in some embodiments, the other stereodefined linkages are common (both with regards the R vs S and position) between the different members of a library. In some embodiments the background internucleoside linkages (i.e. internucleoside linkages other than those in the stereodefined internucleoside motif), may be all R, such as all Rp, or all S, such as all Sp. Oligonucleotides which, other that the stereodefined internucleoside linkage motifs are all S/Sp or are all R/Rp are referred to as having a stereouniform background.

It will also be recognized that in some embodiments the parent oligonucleotide is at least partially stereodefined, such as may be a stereodefined oligonucleotide identified by a previous optimization, and other than the modification of the stereodefined internucleoside motif, the child oligonucleotides may retain one or more stereodefined internucleoside linkages present in the parent oligonucleotide. In some such embodiments the parent oligonucleotide may be a full stereodefined oligonucleotide.

Combinatorial Discovery Methods

The invention relates to methods of identifying improved stereodefined variants of a parent oligonucleotide, employing sub-libraries. The various alternative methods of the invention may be used in parallel or in series, or iteratively.

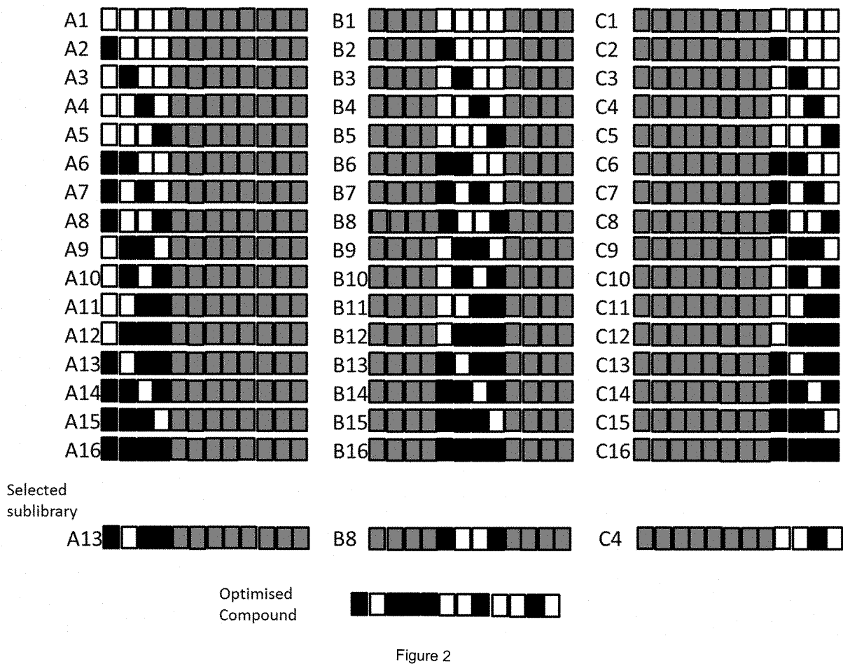

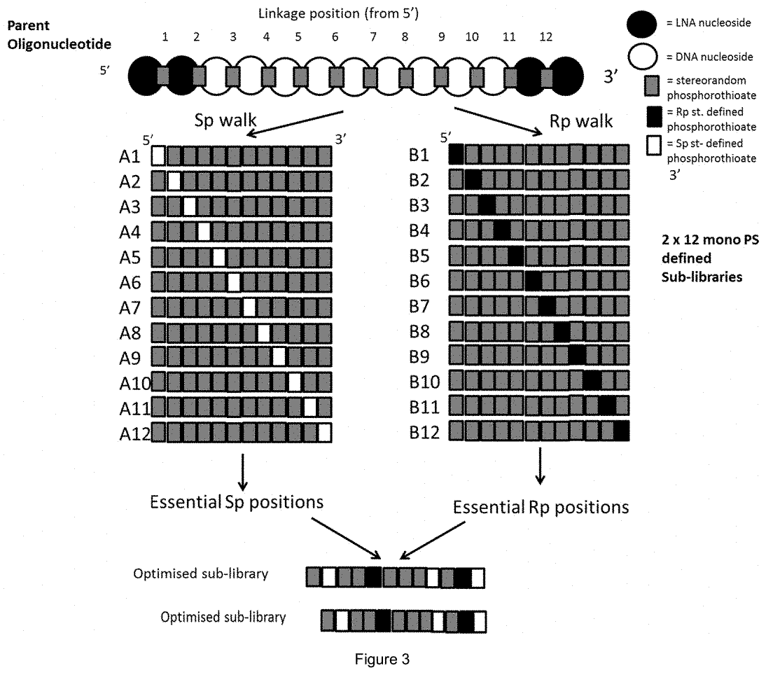

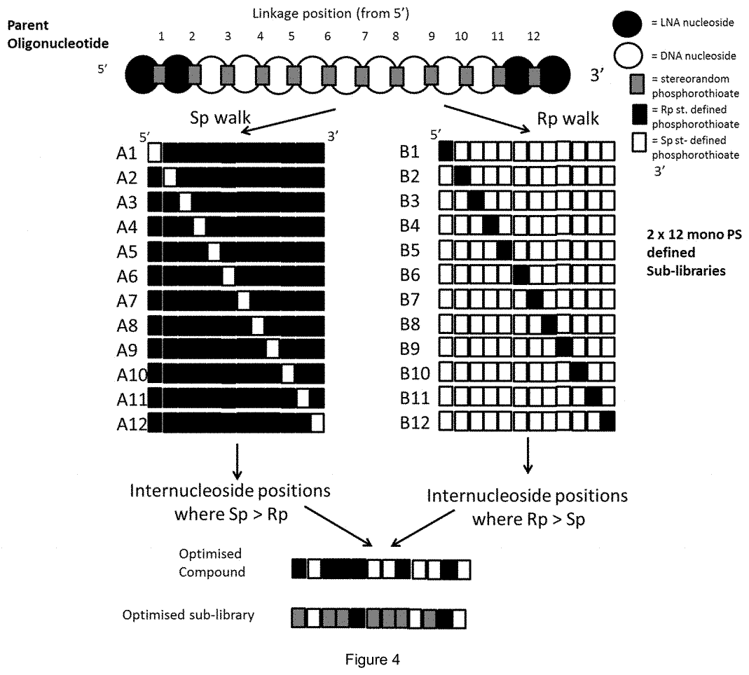

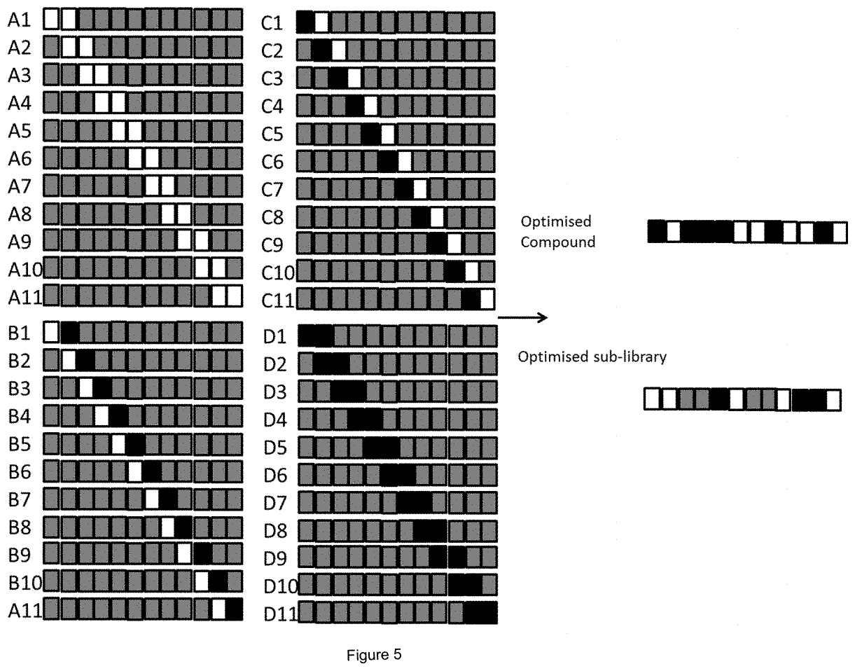

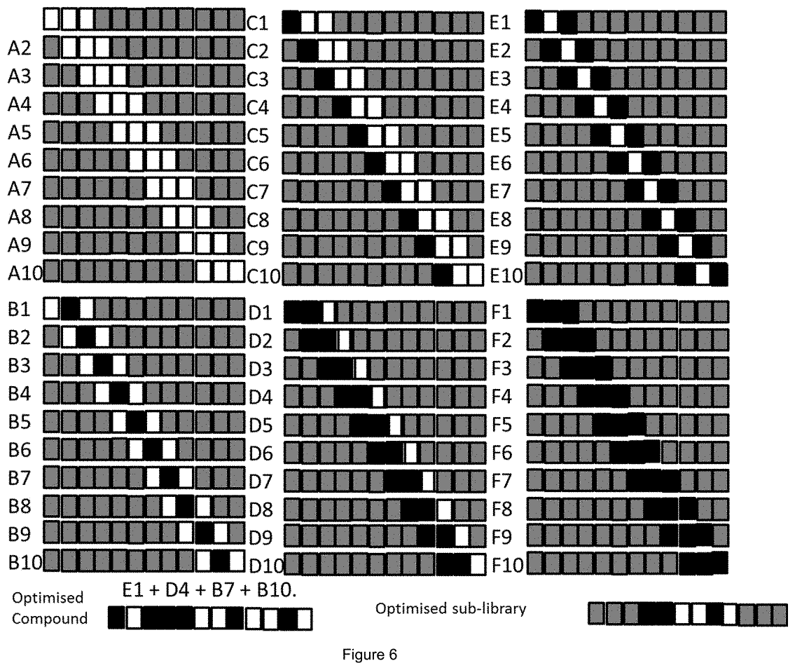

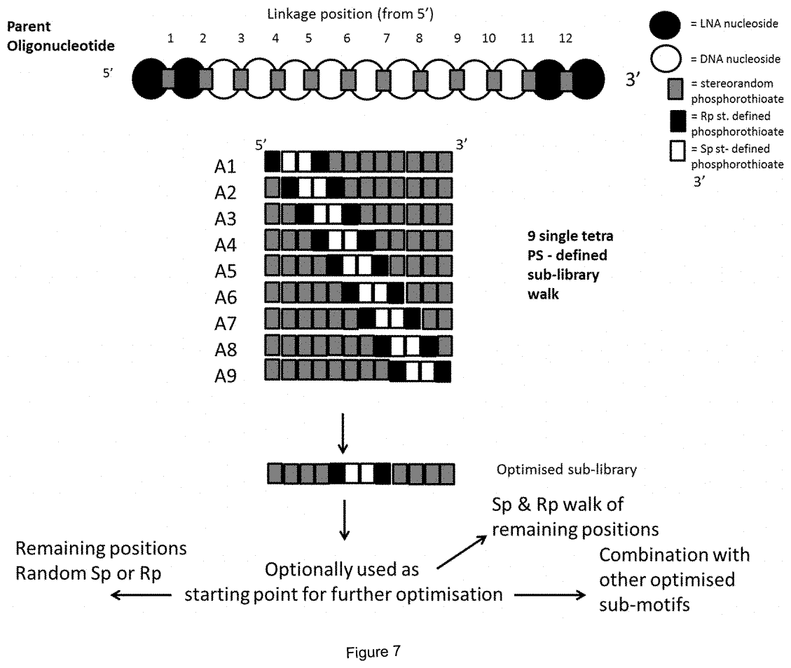

By way of example, in some embodiments, as illustrated in FIG. 2, multiple independent sub-motifs of are optimized in parallel, and the information for each preferred sub-motif obtained from multiple libraries may then be combined.

It is also envisages that an initial library screen may be a oligonucleotide walk to identify essential positions where one of the alternative diastereoisomers is either essential or preferred. In conjunction with this initial library screen, a further library screen may be performed to optimize another region of the oligonucleotide, such a further library screen may be performed in parallel and the preferred motif identified combined with the essential or preferred stereodefined internucleoside linkages identified in the first library in a subsequent step, or the oligonucleotide walk is first performed and the preferred variants identified therefrom are subsequently used as the parent oligonucleotide for one or more subsequent motif optimization methods, wherein the essential or preferred stereodefined internucleoside linkages identified from the initial library screen are retained in the library members in the subsequent motif optimization steps.

Modified Internucleoside Linkage

The term “modified internucleoside linkage” is defined as generally understood by the skilled person as linkages other than phosphodiester (PO) linkages, that covalently couples two nucleosides together. Nucleotides with modified internucleoside linkage are also termed “modified nucleotides”. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide of the invention, for example within the gap region of a gapmer oligonucleotide, as well as in regions of modified nucleosides.

In some embodiments the internucleoside linkage comprises sulphur (S), such as a phosphorothioate internucleoside linkage.

A phosphorothioate internucleoside linkage is particularly useful due to nuclease resistance, beneficial pharmakokinetics and ease of manufacture. In some embodiments at least 50% of the internucleoside linkages in the oligonucleotide, or contiguous nucleotide sequence thereof, are phosphorothioate, such as at least 60%, such as at least 70%, such as at least 80 or such as at least 90% of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate.

Other internucleoside linkages are disclosed in WO2009/124238 (incorporated herein by reference). In an embodiment the internucleoside linkage is selected from linkers disclosed in WO2007/031091 (incorporated herein by reference). Such as, internucleoside linkage may be selected from —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, 0-PO(OCH₃)0, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, —NR^{H}—CO—NR^{H}—, and/or the internucleoside linker may be selected form the group consisting of: —O—CO—O—, —O—CO—NR^{H}—, —NR^{H}—CO—CH₂—, —O—CH₂—CO—NR^{H}—, —O—CH₂—CH₂—NR^{H}—, —CO—NR^{H}—CH₂—, —CH₂—NR^{H}CO—, —O—CH₂—CH₂—S—, —S—CH₂—CH₂—O—, —S—CH₂—CH₂—S—, —CH₂—SO₂—CH₂—, —CH₂—CO—NR^{H}—, —O—CH₂—CH₂—NR^{H}—CO—, —CH₂—NCH₃—O—CH₂—, where R^{H} is selected from hydrogen and C1-4-alkyl.

Nuclease resistant linkages, such as phosphothioate linkages, are particularly useful in oligonucleotide regions capable of recruiting nuclease when forming a duplex with the target nucleic acid, such as region G for gapmers, or the non-modified nucleoside region of headmers and tailmers. Phosphorothioate linkages may, however, also be useful in non-nuclease recruiting regions and/or affinity enhancing regions such as regions F and F′ for gapmers, or the modified nucleoside region of headmers and tailmers.

Each of the design regions may however comprise internucleoside linkages other than phosphorothioate, such as phosphodiester linkages, in particularly in regions where modified nucleosides, such as LNA, protect the linkage against nuclease degradation. Inclusion of phosphodiester linkages, such as one or two linkages, particularly between or adjacent to modified nucleoside units (typically in the non-nuclease recruiting regions) can modify the bioavailability and/or bio-distribution of an oligonucleotide—see WO2008/113832, incorporated herein by reference.

In an embodiment all the internucleoside linkages in the oligonucleotide are phosphorothioates Advantageously, all the internucleoside linkages in the oligonucleotide, or the contiguous nucleotide sequence thereof, are phosphorothioate linkages. In some embodiments all the internucleoside linkages of the oligonucleotide or contiguous nucleotide sequence thereof are phosphorothioate, optionally with 1, 2 or 3 phosphodiester linkages.

Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In a some embodiments the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

Modified Oligonucleotide

The term modified oligonucleotide describes an oligonucleotide comprising one or more sugar-modified nucleosides and/or modified internucleoside linkages. The term “chimeric” oligonucleotide is a term that has been used in the literature to describe oligonucleotides with modified nucleosides and DNA or RNA nucleosides, or oligonucleotides which comprise more than one type of sugar modified nucleosides (e.g. LNA and 2′substituted such as 2′-O-MOE nucleosides. The oligonucleotide or contiguous nucleotide sequence thereof may form a chimeric oligonucleotide.

Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the number of nucleotides in percent of a contiguous nucleotide sequence in a nucleic acid molecule (e.g. oligonucleotide) which, at a given position, are complementary to (i.e. form Watson Crick base pairs with) a contiguous nucleotide sequence, at a given position of a separate nucleic acid molecule (e.g. the target nucleic acid). The percentage is calculated by counting the number of aligned bases that form pairs between the two sequences (when aligned with the target sequence 5′-3′ and the oligonucleotide sequence from 3′-5′), dividing by the total number of nucleotides in the oligonucleotide and multiplying by 100. In such a comparison a nucleobase/nucleotide which does not align (form a base pair) is termed a mismatch. Preferably, insertions and deletions are not allowed in the calculation of % complementarity of a contiguous nucleotide sequence.

The term “fully complementary”, refers to 100% complementarity.

Identity

The term “Identity” as used herein, refers to the number of nucleotides in percent of a contiguous nucleotide sequence in a nucleic acid molecule (e.g. oligonucleotide) which, at a given position, are identical to (i.e. in their ability to form Watson Crick base pairs with the complementary nucleoside) a contiguous nucleotide sequence, at a given position of a separate nucleic acid molecule (e.g. the target nucleic acid). The percentage is calculated by counting the number of aligned bases that are identical between the two sequences dividing by the total number of nucleotides in the oligonucleotide and multiplying by 100. Percent Identity=(Matches×100)/Length of aligned region. Preferably, insertions and deletions are not allowed in the calculation of % complementarity of a contiguous nucleotide sequence.

Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RT ln(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

Target Nucleic Acid

The target nucleic acid may be a mammalian, such as a human RNA, such as a mRNA, and pre-mRNA, a mature mRNA or a cDNA sequence.

For in vivo or in vitro application, the oligonucleotides referred to herein, such as the oligonuceltoides identified by the method of the invention are typically capable of inhibiting the expression of the target nucleic acid in a cell which is expressing the target nucleic acid. In some embodiments, the target nucleic acid is a Hif1alpha encoding nucleic acid. In some embodiments the target nucleic acid is an ApoB encoding nucleic acid.

The contiguous sequence of nucleobases of antisense oligonucleotides are fully complementary to the target nucleic acid, as measured across the length of the oligonucleotide, optionally with the exception of one or two mismatches, and optionally excluding nucleotide based linker regions which may link the oligonucleotide to an optional functional group such as a conjugate, or other non-complementary terminal nucleotides (e.g. region D′ or D″). The target nucleic acid may, in some embodiments, be a RNA or DNA, such as a messenger RNA, such as a mature mRNA or a pre-mRNA.

Antisense oligonucleotides therefore comprise a contiguous nucleotide sequence which is complementary to or hybridizes to the target nucleic acid, such as a sub-sequence of the target nucleic acid.

Antisense oligonucleotides therefore may comprise a contiguous nucleotide sequence of at least 8 nucleotides which is complementary to or hybridizes to a target sequence present in the target nucleic acid molecule. The contiguous nucleotide sequence (and therefore the target sequence) comprises of at least 8 contiguous nucleotides, such as 9, 10, 11, 12, 13, 14, 15, 16, 17, 18, 19, 20, 21, 22, 23, 24, 25, 26, 27, 28, 29 or 30 contiguous nucleotides, such as from 12-25, such as from 14-18 contiguous nucleotides.

Target Cell

The term a “target cell” as used herein refers to a cell which is expressing the target nucleic acid. In some embodiments the target cell may be in vivo or in vitro. In some embodiments the target cell is a mammalian cell such as a rodent cell, such as a mouse cell or a rat cell, or a primate cell such as a monkey cell or a human cell.

Modulation of Expression

The term “modulation of expression” as used herein is to be understood as an overall term for an oligonucleotide's ability to alter the amount of the target nucleic acid when compared to the amount of of the target nucleic acid before administration of the oligonucleotide. Alternatively modulation of expression may be determined by reference to a control experiment. It is generally understood that the control is an individual or target cell treated with a saline composition or an individual or target cell treated with a non-targeting oligonucleotide (mock).

One type of modulation is an oligonucleotide's ability to inhibit, down-regulate, reduce, suppress, remove, stop, block, prevent, lessen, lower, avoid or terminate expression of the target nucleic acid e.g. by degradation of mRNA or blockage of transcription. Another type of modulation is an oligonucleotide's ability to restore, increase or enhance expression of the target nuclic acid, e.g. by repair of splice sites or prevention of splicing or removal or blockage of inhibitory mechanisms such as microRNA repression.

High Affinity Modified Nucleosides

A high affinity modified nucleoside is a modified nucleotide which, when incorporated into the oligonucleotide enhances the affinity of the oligonucleotide for its complementary target, for example as measured by the melting temperature (T^{m}). A high affinity modified nucleoside of the present invention preferably result in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

Sugar Modifications

The oligomer of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

2′ Sugar Modified Nucleosides.

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

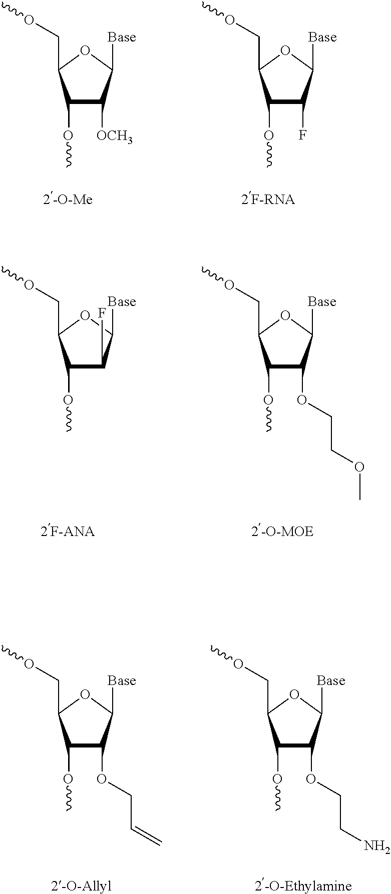

In relation to the present invention 2′ substituted does not include 2′ bridged molecules like LNA.

Locked Nucleic Acid Nucleosides (LNA).

An “LNA nucleoside” is 2′-modified nucleoside which comprises a biradical linking the C2′ and C4′ of the ribose sugar ring of said nucleoside (a “2′-4′ bridge”), which restricts or locks the conformation of the ribose ring. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76,

Seth et al. J. Org. Chem. 2010, Vol 75(5) pp. 1569-81, and Mitsuoka et al., Nucleic Acids Research 2009, 37(4), 1225-1238.

In some embodiments, the sugar modified nucleoside(s) or the LNA nucleoside(s) of the oligomer of the invention has a general structure of the formula I or II:

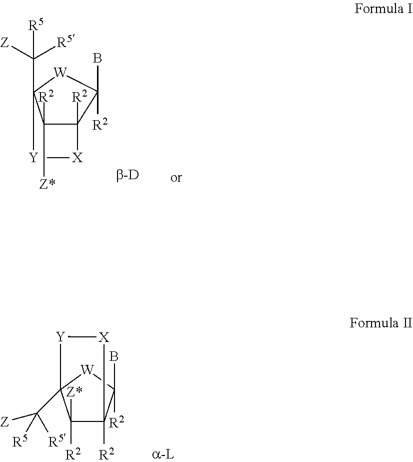

wherein W is selected from —O—, —S—, —N(R^{a})—, —C(R^{a}R^{b})—, such as, in some embodiments —O—;

B designates a nucleobase or modified nucleobase moiety;

Z designates an internucleoside linkage to an adjacent nucleoside, or a 5′-terminal group;

Z* designates an internucleoside linkage to an adjacent nucleoside, or a 3′-terminal group;

X designates a group selected from the list consisting of —C(R^{a}R^{b})—, —C(R^{a})═C(R^{b})—, —C(R^{a})═N—, —O—, —Si(R^{a})₂—, —S—, —SO₂—, —N(R^{a})—, and >C═Z
In some embodiments, X is selected from the group consisting of: —O—, —S—, NH—, NR^{a}R^{b}, —CH₂—, CR^{a}R^{b}, —C(═CH₂)—, and —C(═CR^{a}R^{b})—
In some embodiments, X is —O—

Y designates a group selected from the group consisting of —C(R^{a}R^{b})—, —C(R^{a})═C(R^{b}), —C(R^{a})═N—, —O—, —S(R^{a})₂—, —S—, —SO₂—, —N(R^{a})—, and >C═Z
In some embodiments, Y is selected from the group consisting of: —CH₂—, —C(R^{a}R^{b}), —CH₂CH₂—, —C(R^{a}R^{b})—C(R^{a}R^{b})—, —CH₂CH₂CH₂—, —C(R^{a}R^{b})C(R^{a}R^{b})C(R^{a}R^{b})—, —C(R^{a})═C(R^{b})—, and —C(R^{a})═N—
In some embodiments, Y is selected from the group consisting of: —CH₂—, —CHR^{a}—, —CHCH₃—, CR^{a}R^{b}—

or —X—Y— together designate a bivalent linker group (also referred to as a radicle) together designate a bivalent linker group consisting of 1, 2, 3 or 4 groups/atoms selected from the group consisting of —C(R^{a}R^{b})—, —C(R^{a})═C(R^{b})—, —C(R^{a})═N—, —O—, —Si(R^{a})₂—, —S—, —SO₂—, —N(R^{a})—, and >C═Z,
In some embodiments, —X—Y— designates a biradicle selected from the groups consisting of: —X—CH₂—, —X—CR^{a}R^{b}—, —X—CHR^{a}—, —X—C(HCH₃)⁻, —O—Y—, —O—CH₂—, —S—CH₂—, —NH—CH₂—, —O—CHCH₃—, —CH₂—O—CH₂, —O—CH(CH₃CH₃)—, —O—CH₂—CH₂—, OCH₂—CH₂—CH₂—, —O—CH₂OCH₂—, —O—NCH₂—, —C(═CH₂)—CH₂—, —NR^{a}—CH₂—, N—O—CH₂, —S—CR^{a}R^{b}— and —S—CHR^{a}—.
In some embodiments —X—Y— designates —O—CH₂— or —O—CH(CH₃)—.

wherein Z is selected from —O—, —S—, and —N(R^{a})—,

and R^{a} and, when present R^{b}, each is independently selected from hydrogen, optionally substituted C₁₋₆-alkyl, optionally substituted C₂₋₆-alkenyl, optionally substituted C₂₋₆-alkynyl, hydroxy, optionally substituted C₁₋₆-alkoxy, C₂₋₆-alkoxyalkyl, C₂₋₆-alkenyloxy, carboxy, C₁₋₆-alkoxycarbonyl, C₁₋₆-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, where aryl and heteroaryl may be optionally substituted and where two geminal substituents R^{a} and R^{b} together may designate optionally substituted methylene (═CH₂), wherein for all chiral centers, asymmetric groups may be found in either R or S orientation.

wherein R¹, R², R³, R⁵ and R^{5*} are independently selected from the group consisting of: hydrogen, optionally substituted C₁₋₆-alkyl, optionally substituted C₂₋₆-alkenyl, optionally substituted C₂₋₆-alkynyl, hydroxy, C₁₋₆-alkoxy, C₂₋₆-alkoxyalkyl, C₂₋₆-alkenyloxy, carboxy, C₁₋₆-alkoxycarbonyl, C₁₋₆-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, where aryl and heteroaryl may be optionally substituted, and where two geminal substituents together may designate oxo, thioxo, imino, or optionally substituted methylene.
In some embodiments R¹, R², R³, R⁵ and R^{5*} are independently selected from C₁₋₆ alkyl, such as methyl, and hydrogen.
In some embodiments R¹, R², R³, R⁵ and R^{5*} are all hydrogen.
In some embodiments R¹, R², R³, are all hydrogen, and either R⁵ and R^{5*} is also hydrogen and the other of R⁵ and R^{5*}is other than hydrogen, such as C₁₋₆ alkyl such as methyl.
In some embodiments, R^{a} is either hydrogen or methyl. In some embodiments, when present, R^{b} is either hydrogen or methyl.
In some embodiments, one or both of R^{a} and R^{b} is hydrogen
In some embodiments, one of R^{a} and R^{b} is hydrogen and the other is other than hydrogen
In some embodiments, one of R^{a} and R^{b} is methyl and the other is hydrogen
In some embodiments, both of R^{a} and R^{b} are methyl.

In some embodiments, the biradicle —X—Y— is —O—CH₂—, W is O, and all of R¹, R², R³, R⁵ and R^{5*} are all hydrogen. Such LNA nucleosides are disclosed in WO99/014226, WO00/66604, WO98/039352 and WO2004/046160 which are all hereby incorporated by reference, and include what are commonly known as beta-D-oxy LNA and alpha-L-oxy LNA nucleosides.

In some embodiments, the biradicle —X—Y— is —S—CH₂—, W is O, and all of R¹, R², R³, R⁵ and R^{5*} are all hydrogen. Such thio LNA nucleosides are disclosed in WO99/014226 and WO2004/046160 which are hereby incorporated by reference.

In some embodiments, the biradicle —X—Y— is —NH—CH₂—, W is O, and all of R¹, R², R³, R⁵ and R^{5*} are all hydrogen. Such amino LNA nucleosides are disclosed in WO99/014226 and WO2004/046160 which are hereby incorporated by reference.

In some embodiments, the biradicle —X—Y— is —O—CH₂—CH₂— or —O—CH₂—CH₂—CH₂—, W is O, and all of R¹, R², R³, R⁵ and R^{5*} are all hydrogen. Such LNA nucleosides are disclosed in WO00/047599 and Morita et al, Bioorganic & Med. Chem. Lett. 12 73-76, which are hereby incorporated by reference, and include what are commonly known as 2′-O-4′C-ethylene bridged nucleic acids (ENA).

In some embodiments, the biradicle —X—Y— is —O—CH₂—, W is O, and all of R¹, R², R³, and one of R⁵ and R^{5*} are hydrogen, and the other of R⁵ and R^{5*} is other than hydrogen such as C₁₋₆ alkyl, such as methyl. Such 5′ substituted LNA nucleosides are disclosed in WO2007/134181 which is hereby incorporated by reference.

In some embodiments, the biradicle —X—Y— is —O—CR^{a}R^{b}—, wherein one or both of R^{a} and R^{b} are other than hydrogen, such as methyl, W is O, and all of R¹, R², R³, and one of R⁵ and R^{5*} are hydrogen, and the other of R⁵ and R^{5*} is other than hydrogen such as C₁₋₆ alkyl, such as methyl. Such bis modified LNA nucleosides are disclosed in WO2010/077578 which is hereby incorporated by reference.

In some embodiments, the biradicle —X—Y— designate the bivalent linker group —O—CH(CH₂OCH₃)— (2′O-methoxyethyl bicyclic nucleic acid—Seth at al., 2010, J. Org. Chem. Vol 75(5) pp. 1569-81). In some embodiments, the biradicle —X—Y— designate the bivalent linker group —O—CH(CH₂CH₃)— (2′O-ethyl bicyclic nucleic acid—Seth at al., 2010, J. Org. Chem. Vol 75(5) pp. 1569-81). In some embodiments, the biradicle —X—Y— is —O—CHR^{a}—, W is O, and all of R¹, R², R³, R⁵ and R^{5*} are all hydrogen. Such 6′ substituted LNA nucleosides are disclosed in WO10036698 and WO07090071 which are both hereby incorporated by reference.

In some embodiments, the biradicle —X—Y— is —O—CH(CH₂OCH₃)—, W is O, and all of R¹, R², R³, R⁵ and R^{5*} are all hydrogen. Such LNA nucleosides are also known as cyclic MOEs in the art (cMOE) and are disclosed in WO07090071.

In some embodiments, the biradicle —X—Y— designate the bivalent linker group —O—CH(CH₃)—.—in either the R- or S-configuration. In some embodiments, the biradicle —X—Y— together designate the bivalent linker group —O—CH₂—O—CH₂— (Seth at al., 2010, J. Org. Chem). In some embodiments, the biradicle —X—Y— is —O—CH(CH₃)—, W is O, and all of R¹, R², R³, R⁵ and R^{5*} are all hydrogen. Such 6′ methyl LNA nucleosides are also known as cET nucleosides in the art, and may be either (S)cET or (R)cET stereoisomers, as disclosed in WO07090071 (beta-D) and WO2010/036698 (alpha-L) which are both hereby incorporated by reference).

In some embodiments, the biradicle —X—Y— is —O—CR^{a}R^{b}—, wherein in neither R^{a} or R^{b} is hydrogen, W is O, and all of R¹, R², R³, R⁵ and R^{5*} are all hydrogen. In some embodiments, R^{a} and R^{b} are both methyl. Such 6′ di-substituted LNA nucleosides are disclosed in WO 2009006478 which is hereby incorporated by reference.

In some embodiments, the biradicle —X—Y— is —S—CHR^{a}—, W is O, and all of R¹, R², R³, R⁵ and R^{5*} are all hydrogen. Such 6′ substituted thio LNA nucleosides are disclosed in WO11156202 which is hereby incorporated by reference. In some 6′ substituted thio LNA embodiments R^{a} is methyl.

In some embodiments, the biradicle —X—Y— is —C(═CH2)-C(R^{a}R^{b})—, such as —C(═CH₂)—CH₂—, or —C(═CH₂)—CH(CH₃)—W is O, and all of R¹, R², R³, R⁵ and R^{5*} are all hydrogen. Such vinyl carbo LNA nucleosides are disclosed in WO08154401 and WO09067647 which are both hereby incorporated by reference.

In some embodiments the biradicle —X—Y— is —N(—OR^{a})—, W is O, and all of R¹, R², R³, R⁵ and R^{5*} are all hydrogen. In some embodiments R^{a} is C₁₋₆ alkyl such as methyl. Such LNA nucleosides are also known as N substituted LNAs and are disclosed in WO2008/150729 which is hereby incorporated by reference. In some embodiments, the biradicle —X—Y— together designate the bivalent linker group —O—NR^{a}—CH₃— (Seth at al., 2010, J. Org. Chem). In some embodiments the biradicle —X—Y— is —N(R^{a})—, W is O, and all of R¹, R², R³, R⁵ and R^{5*} are all hydrogen. In some embodiments R^{a} is C₁₋₆ alkyl such as methyl.

In some embodiments, one or both of R⁵ and R^{5*} is hydrogen and, when substituted the other of R⁵ and R^{5*} is C₁₋₆ alkyl such as methyl. In such an embodiment, R¹, R², R³, may all be hydrogen, and the biradicle —X—Y— may be selected from —O—CH2- or —O—C(HCR^{a})—, such as —O—C(HCH3)—.

In some embodiments, the biradicle is —CR^{a}R^{b}—O—CR^{a}R^{b}—, such as CH₂—O—CH₂—, W is O and all of R¹, R², R³, R⁵ and R^{5*} are all hydrogen. In some embodiments R^{a} is C₁₋₆alkyl such as methyl. Such LNA nucleosides are also known as conformationally restricted nucleotides (CRNs) and are disclosed in WO2013036868 which is hereby incorporated by reference.

In some embodiments, the biradicle is —O—CR^{a}R^{b}—O—CR^{a}R—, such as O—CH2-O—CH₂—, W is O and all of R¹, R², R³, R⁵ and R^{5*} are all hydrogen. In some embodiments R^{a} is C₁₋₆alkyl such as methyl. Such LNA nucleosides are also known as COC nucleotides and are disclosed in Mitsuoka et al., Nucleic Acids Research 2009 37(4), 1225-1238, which is hereby incorporated by reference.

It will be recognized than, unless specified, the LNA nucleosides may be in the beta-D or alpha-L stereoisoform.

Certain examples of LNA nucleosides are presented in Scheme 1.

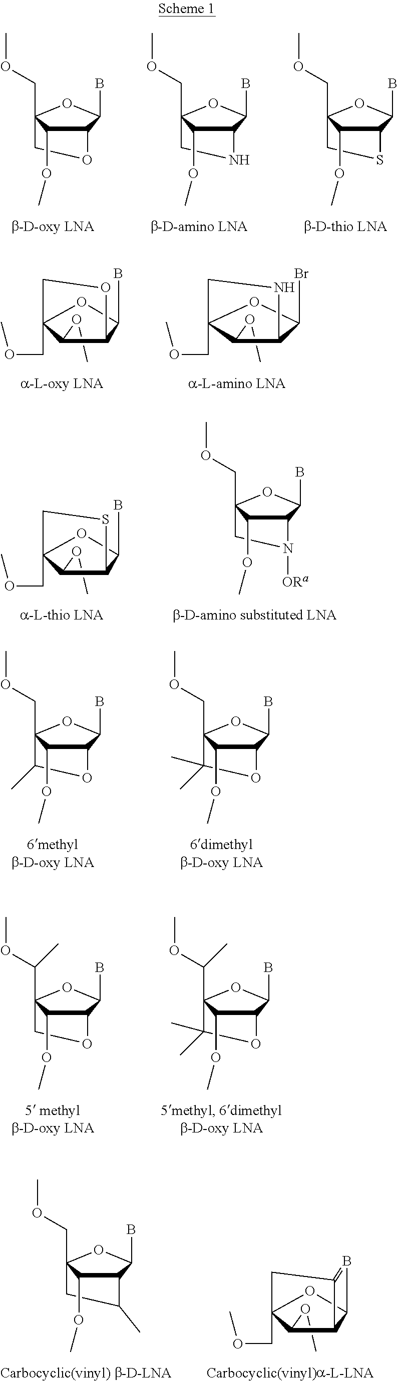

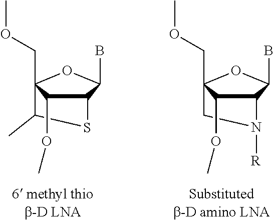

As illustrated in the examples, in some embodiments of the invention the LNA nucleosides in the oligonucleotides are beta-D-oxy-LNA nucleosides.

Nuclease Mediated Degradation

Nuclease mediated degradation refers to an oligonucleotide capable of mediating degradation of a complementary nucleotide sequence when forming a duplex with such a sequence.

In some embodiments, the oligonucleotide may function via nuclease mediated degradation of the target nucleic acid, where the oligonucleotides of the invention are capable of recruiting a nuclease, particularly and endonuclease, preferably endoribonuclease (RNase), such as RNase H. Examples of oligonucleotide designs which operate via nuclease mediated mechanisms are oligonucleotides which typically comprise a region of at least 5 or 6 consecutive DNA nucleosides and are flanked on one side or both sides by affinity enhancing nucleosides, for example gapmers, headmers and tailmers.

RNase H Activity and Recruitment

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO01/23613 (hereby incorporated by reference).

Gapmer Oligonucelotides and Gapmer Designs

The antisense oligonucleotide of the invention, or contiguous nucleotide sequence thereof may be a gapmer. The antisense gapmers are commonly used to inhibit a target nucleic acid via RNase H mediated degradation. A gapmer oligonucleotide comprises at least three distinct structural regions a 5′-flank, a gap and a 3′-flank, F-G-F′ in the ‘5->3’ orientation. The “gap” region (G) comprises a stretch of contiguous DNA nucleotides which enable the oligonucleotide to recruit RNase H. The Gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides, and are positioned adjacent to a sugar modified nucleoside of the 5′ (F) or 3′ (F′) region respectively. The flanks may further defined by having at least one sugar modified nucleoside at the end most distant from the gap region, i.e. at the 5′ end of the 5′ flank and at the 3′ end of the 3′ flank.

Regions F-G-F′ form a contiguous nucleotide sequence. Antisense oligonucleotides of the invention, or the contiguous nucleotide sequence thereof, may comprise a gapmer region of formula F-G-F′.

The overall length of the gapmer design F-G-F′ may be, for example 12 to 32 nucleosides, such as 13 to 24, such as 14 to 22 nucleosides, Such as from 14 to 17, such as 16 to 18 nucleosides.

By way of example, the gapmer oligonucleotide of the present invention can be represented by the following formulae:

F₁₋₈-G₅₋₁₆-F′₁₋₈, such as

F₁₋₈-G₇₋₁₆-F′₂₋₈

with the proviso that the overall length of the gapmer regions F-G-F′ is at least 12, such as at least 14 nucleotides in length.

Regions F, G and F′ are further defined below and can be incorporated into the F-G-F′ formula.

Gapmer Region G

Region G (gap region) of the gapmer is a region of nucleosides which enables the oligonucleotide to recruit RNaseH, such as human RNase H1, typically DNA nucleosides. RNaseH is a cellular enzyme which recognizes the duplex between DNA and RNA, and enzymatically cleaves the RNA molecule. Suitably gapmers may have a gap region (G) of at least 5 or 6 contiguous DNA nucleosides, such as 5-16 contiguous DNA nucleosides, such as 6-15 contiguous DNA nucleosides, such as 7-14 contiguous DNA nucleosides, such as 8-12 contiguous DNA nucleotides, such as 8-12 contiguous DNA nucleotides in length. The gap region G may, in some embodiments consist of 6, 7, 8, 9, 10, 11, 12, 13, 14, 15 or 16 contiguous DNA nucleosides.

In some embodiments the gap region G may consist of 6, 7, 8, 9, 10, 11, 12, 13, 14, 15 or 16 contiguous phosphorothioate linked DNA nucleosides. In some embodiments, all internucleoside linkages in the gap are phosphorothioate linkages.

Whilst traditional gapmers have a DNA gap region, there are numerous examples of modified nucleosides which allow for RNaseH recruitment when they are used within the gap region. Modified nucleosides which have been reported as being capable of recruiting RNaseH when included within a gap region include, for example, alpha-L-LNA, C4′ alkylated DNA (as described in PCT/EP2009/050349 and Vester et al., Bioorg. Med. Chem. Lett. 18 (2008) 2296-2300, both incorporated herein by reference), arabinose derived nucleosides like ANA and 2′F-ANA (Mangos et al. 2003 J. AM. CHEM. SOC. 125, 654-661), UNA (unlocked nucleic acid) (as described in Fluiter et al., Mol. Biosyst., 2009, 10, 1039 incorporated herein by reference). UNA is unlocked nucleic acid, typically where the bond between C2 and C3 of the ribose has been removed, forming an unlocked “sugar” residue. 5′ substituted DNA nucleosides, such as 5′methyl DNA nucleoside have been reported for use in DNA gap regions (EP 2 742 136). The modified nucleosides used in such gapmers may be nucleosides which adopt a 2′ endo (DNA like) structure when introduced into the gap region, i.e. modifications which allow for RNaseH recruitment). In some embodiments the DNA Gap region (G) described herein may optionally contain 1 to 3 sugar modified nucleosides which adopt a 2′ endo (DNA like) structure when introduced into the gap region.

Region G “Gap-Breaker”

Alternatively, there are numerous reports of the insertion of a modified nucleoside which confers a 3′ endo conformation into the gap region of gapmers, whilst retaining some RNaseH activity. Such gapmers with a gap region comprising one or more 3′endo modified nucleosides are referred to as “gap-breaker” or “gap-disrupted” gapmers, see for example WO2013/022984. Gap-breaker oligonucleotides retain sufficient region of DNA nucleosides within the gap region to allow for RNaseH recruitment. The ability of gapbreaker oligonucleotide design to recruit RNaseH is typically sequence or even compound specific—see Rukov et al. 2015 Nucl. Acids Res. Vol. 43 pp. 8476-8487, which discloses “gapbreaker” oligonucleotides which recruit RNaseH which in some instances provide a more specific cleavage of the target RNA. Modified nucleosides used within the gap region of gap-breaker oligonucleotides may for example be modified nucleosides which confer a 3′endo confirmation, such 2′-O-methyl (OMe) or 2′-O-MOE (MOE) nucleosides, or beta-D LNA nucleosides (the bridge between C2′ and C4′ of the ribose sugar ring of a nucleoside is in the beta conformation), such as beta-D-oxy LNA or ScET nucleosides.

As with gapmers containing region G described above, the gap region of gap-breaker or gap-disrupted gapmers, have a DNA nucleosides at the 5′ end of the gap (adjacent to the 3′ nucleoside of region F), and a DNA nucleoside at the 3′ end of the gap (adjacent to the 5′ nucleoside of region F′). Gapmers which comprise a disrupted gap typically retain a region of at least 3 or 4 contiguous DNA nucleosides at either the 5′ end or 3′ end of the gap region.

Exemplary designs for gap-breaker oligonucleotides include

F₁₋₈-[D₃₋₄-E₁-D₃₋₄]-F′₁₋₈

F₁₋₈-[D₁₋₄-E₁-D₃₋₄]-F′₁₋₈

F₁₋₈-[D₃₋₄-E₁-D₁₋₄]-F′₁₋₈

wherein region G is within the brackets [Dₙ-Eᵣ-Dₘ], D is a contiguous sequence of DNA nucleosides, E is a modified nucleoside (the gap-breaker or gap-disrupting nucleoside), and F and F′ are the flanking regions as defined herein, and with the proviso that the overall length of the gapmer regions F-G-F′ is at least 12, such as at least 14 nucleotides in length.

In some embodiments, region G of a gap disrupted gapmer comprises at least 6 DNA nucleosides, such as 6, 7, 8, 9, 10, 11, 12, 13, 14, 15 or 16 DNA nucleosides. As described above, the DNA nucleosides may be contiguous or may optionally be interspersed with one or more modified nucleosides, with the proviso that the gap region G is capable of mediating RNaseH recruitment.

Gapmer Flanking Regions, F and F′

Region F is positioned immediately adjacent to the 5′ DNA nucleoside of region G. The 3′ most nucleoside of region F is a sugar modified nucleoside, such as a high affinity sugar modified nucleoside, for example a 2′ substituted nucleoside, such as a MOE nucleoside, or an LNA nucleoside.

Region F′ is positioned immediately adjacent to the 3′ DNA nucleoside of region G. The 5′ most nucleoside of region F′ is a sugar modified nucleoside, such as a high affinity sugar modified nucleoside, for example a 2′ substituted nucleoside, such as a MOE nucleoside, or an LNA nucleoside.

Region F is 1-8 contiguous nucleotides in length, such as 2-6, such as 3-4 contiguous nucleotides in length. Advantageously the 5′ most nucleoside of region F is a sugar modified nucleoside. In some embodiments the two 5′ most nucleoside of region F are sugar modified nucleoside. In some embodiments the 5′ most nucleoside of region F is an LNA nucleoside. In some embodiments the two 5′ most nucleoside of region F are LNA nucleosides. In some embodiments the two 5′ most nucleoside of region F are 2′ substituted nucleoside nucleosides, such as two 3′ MOE nucleosides. In some embodiments the 5′ most nucleoside of region F is a 2′ substituted nucleoside, such as a MOE nucleoside.

Region F′ is 2-8 contiguous nucleotides in length, such as 3-6, such as 4-5 contiguous nucleotides in length. Advantageously, embodiments the 3′ most nucleoside of region F′ is a sugar modified nucleoside. In some embodiments the two 3′ most nucleoside of region F′ are sugar modified nucleoside. In some embodiments the two 3′ most nucleoside of region F′ are LNA nucleosides. In some embodiments the 3′ most nucleoside of region F′ is an LNA nucleoside. In some embodiments the two 3′ most nucleoside of region F′ are 2′ substituted nucleoside nucleosides, such as two 3′ MOE nucleosides. In some embodiments the 3′ most nucleoside of region F′ is a 2′ substituted nucleoside, such as a MOE nucleoside.

It should be noted that when the length of region F or F′ is one, it is advantageously an LNA nucleoside.

In some embodiments, region F and F′ independently consists of or comprises a contiguous sequence of sugar modified nucleosides. In some embodiments, the sugar modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In some embodiments, region F and F′ independently comprises both LNA and a 2′ substituted modified nucleosides (mixed wing design).

In some embodiments, all the nucleosides of region F or F′, or F and F′ are LNA nucleosides, such as independently selected from beta-D-oxy LNA, ENA or ScET nucleosides. In some embodiments region F consists of 1-5, such as 2-4, such as 3-4 such as 1, 2, 3, 4 or 5 contiguous LNA nucleosides. In some embodiments, all the nucleosides of region F and F′ are beta-D-oxy LNA nucleosides.

In some embodiments, all the nucleosides of region F or F′, or F and F′ are 2′ substituted nucleosides, such as OMe or MOE nucleosides. In some embodiments region F consists of 1, 2, 3, 4, 5, 6, 7, or 8 contiguous OMe or MOE nucleosides. In some embodiments only one of the flanking regions can consist of 2′ substituted nucleosides, such as OMe or MOE nucleosides. In some embodiments it is the 5′ (F) flanking region that consists 2′ substituted nucleosides, such as OMe or MOE nucleosides whereas the 3′ (F′) flanking region comprises at least one LNA nucleoside, such as beta-D-oxy LNA nucleosides or cET nucleosides. In some embodiments it is the 3′ (F′) flanking region that consists 2′ substituted nucleosides, such as OMe or MOE nucleosides whereas the 5′ (F) flanking region comprises at least one LNA nucleoside, such as beta-D-oxy LNA nucleosides or cET nucleosides.

In some embodiments, all the modified nucleosides of region F and F′ are LNA nucleosides, such as independently selected from beta-D-oxy LNA, ENA or ScET nucleosides, wherein region F or F′, or F and F′ may optionally comprise DNA nucleosides (an alternating flank, see definition of these for more details). In some embodiments, all the modified nucleosides of region F and F′ are beta-D-oxy LNA nucleosides, wherein region F or F′, or F and F′ may optionally comprise DNA nucleosides (an alternating flank, see definition of these for more details).

In some embodiments the 5′ most and the 3′ most nucleosides of region F and F′ are LNA nucleosides, such as beta-D-oxy LNA nucleosides or ScET nucleosides.

In some embodiments, the internucleoside linkage between region F and region G is a phosphorothioate internucleoside linkage. In some embodiments, the internucleoside linkage between region F′ and region G is a phosphorothioate internucleoside linkage. In some embodiments, the internucleoside linkages between the nucleosides of region F or F′, F and F′ are phosphorothioate internucleoside linkages.

LNA Gapmers

An LNA gapmer is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In some embodiments the LNA gapmer is of formula: [LNA]₁₋₅-[region G]-[LNA]₁₋₅, wherein region G is as defined in the Gapmer definition.

MOE Gapmers

A MOE gapmers is a gapmer wherein regions F and F′ consist of MOE nucleosides. In some embodiments the MOE gapmer is of design [MOE]₁₋₈-[Region G]-[MOE]₁₋₈, such as [MOE]₂₋₇-[Region G]₅₋₁₆-[MOE]₂₋₇, such as [MOE]₃₋₆-[Region G]-[MOE]₃₋₆, wherein region G is as defined in the Gapmer definition. MOE gapmers with a 5-10-5 design (MOE-DNA-MOE) have been widely used in the art.

Mixed Wing Gapmers

A mixed wing gapmer is an LNA gapmer wherein one or both of region F and F′ comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units, such as a MOE nucleosides. In some embodiments wherein at least one of region F and F′, or both region F and F′ comprise at least one LNA nucleoside, the remaining nucleosides of region F and F′ are independently selected from the group consisting of MOE and LNA. In some embodiments wherein at least one of region F and F′, or both region F and F′ comprise at least two LNA nucleosides, the remaining nucleosides of region F and F′ are independently selected from the group consisting of MOE and LNA. In some mixed wing embodiments, one or both of region F and F′ may further comprise one or more DNA nucleosides.

Mixed wing gapmer designs are disclosed in WO2008/049085 and WO2012/109395, both of which are hereby incorporated by reference.

Alternating Flank Gapmers

Oligonucleotides with alternating flanks are LNA gapmer oligonucleotides where at least one of the flanks (F or F′) comprises DNA in addition to the LNA nucleoside(s). In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F and/or F′ region are LNA nucleosides.

In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F or F′ region are LNA nucleosides, and the. Flanking regions which comprise both LNA and DNA nucleoside are referred to as alternating flanks, as they comprise an alternating motif of LNA-DNA-LNA nucleosides. Alternating flank LNA gapmers are disclosed in WO2016/127002.

An alternating flank region may comprise up to 3 contiguous DNA nucleosides, such as 1 to 2 or 1 or 2 or 3 contiguous DNA nucleosides.

The alternating flak can be annotated as a series of integers, representing a number of LNA nucleosides (L) followed by a number of DNA nucleosides (D), for example

[L]₁₋₃-[D]₁₋₄-[L]₁₋₃

[L]₁₋₂-[D]₁₋₂-[L]₁₋₂-[D]₁₋₂-[L]₁₋₂

In oligonucleotide designs these will often be represented as numbers such that 2-2-1 represents 5′ [L]₂-[D]₂-[L] 3′, and 1-1-1-1-1 represents 5′ [L]-[D]-[L]-[D]-[L] 3′. The length of the flank (region F and F′) in oligonucleotides with alternating flanks may independently be 3 to 10 nucleosides, such as 4 to 8, such as 5 to 6 nucleosides, such as 4, 5, 6 or 7 modified nucleosides. In some embodiments only one of the flanks in the gapmer oligonucleotide is alternating while the other is constituted of LNA nucleotides. It may be advantageous to have at least two LNA nucleosides at the 3′ end of the 3′ flank (F′), to confer additional exonuclease resistance. Some examples of oligonucleotides with alternating flanks are:

[L]₁₋₅-[D]₁₋₄-[L]₁₋₃-[G]₅₋₁₆-[L]₂₋₆

[L]₁₋₂-[D]₁₋₂-[L]₁₋₂-[D]₁₋₂-[L]₁₋₂-[G]₅₋₁₆-[L]₁₋₂-[D]₁₋₃-[L]₂₋₄

[L]₁₋₅-[G]₅₋₁₆-[L]-[D]-[L]-[D]-[L]₂

with the proviso that the overall length of the gapmer regions F-G-F′ is at least 12, such as at least 14 nucleotides in length.

Totalmers

In some embodiments, all of the nucleosides of the oligonucleotide, or contiguous nucleotide sequence thereof, are sugar modified nucleosides. Such oligonucleotides are referred to as a totalmers herein.

In some embodiments all of the sugar modified nucleosides of a totalmer comprise the same sugar modification, for example they may all be LNA nucleosides, or may all be 2′O-MOE nucleosides. In some embodiments the sugar modified nucleosides of a totalmer may be independently selected from LNA nucleosides and 2′ substituted nucleosides, such as 2′ substituted nucleoside selected from the group consisting of 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleosides. In some embodiments the oligonucleotide comprises both LNA nucleosides and 2′ substituted nucleosides, such as 2′ substituted nucleosides, such as 2′ substituted nucleoside selected from the group consisting of 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleosides. In some embodiments, the oligonucleoitide comprises LNA nucleosides and 2′-O-MOE nucleosides. In some embodiments, the oligonucleotide comprises (S)cET LNA nucleosides and 2′-O-MOE nucleosides.

In some embodiments, all of the nucleosides of the oligonucleotide or contiguous nucleotide sequence thereof are LNA nucleosides, such as beta-D-oxy-LNA nucleosides and/or (S)cET nucleosides. In some embodiments such LNA totalmer oligonucleotides are between 7-12 nucleosides in length (see for example, WO2009/043353). Such short fully LNA oligonucelotides are particularly effective in inhibiting microRNAs.

Various totalmer compounds are highly effective as therapeutic oligomers, particularly when targeting microRNA (antimiRs) or as splice switching oligomers (SSOs).

In some embodiments, the totalmer comprises or consists of at least one XYX or YXY sequence motif, such as a repeated sequence XYX or YXY, wherein X is LNA and Y is an alternative (i.e. non LNA) nucleotide analogue, such as a 2′-OMe RNA unit and 2′-fluoro DNA unit. The above sequence motif may, in some embodiments, be XXY, XYX, YXY or YYX for example.

In some embodiments, the totalmer may comprise or consist of a contiguous nucleotide sequence of between 7 and 24 nucleotides, such as 7, 9, 10, 11, 12, 13, 14, 15, 16, 17, 18, 19, 20, 21, 22 or 23 nucleotides.

In some embodiments, the contiguous nucleotide sequence of the totolmer comprises of at least 30%, such as at least 40%, such as at least 50%, such as at least 60%, such as at least 70%, such as at least 80%, such as at least 90%, such as 95%, such as 100% LNA units. For full LNA compounds, it is advantageous that they are less than 12 nucleotides in length, such as 7-10.

The remaining units may be selected from the non-LNA nucleotide analogues referred to herein in, such those selected from the group consisting of 2′-O-alkyl-RNA unit, 2′-OMe-RNA unit, 2′-amino-DNA unit, 2′-fluoro-DNA unit, LNA unit, PNA unit, HNA unit, INA unit, and a 2′MOE RNA unit, or the group 2′-OMe RNA unit and 2′-fluoro DNA unit.

### Mixmers

The term ‘mixmer’ refers to oligomers which comprise both DNA nucleosides and sugar modified nucleosides, wherein there are insufficient length of contiguous DNA nucleosides to recruit RNaseH. Suitably mixmers may comprise up to 3 or up to 4 contiguous DNA nucleosides. In some embodiments the mixmers comprise alternating regions of sugar modified nucleosides, and DNA nucleosides. By alternating regions of sugar modified nucleosides which form a RNA like (3′endo) conformation when incorporated into the oligonucleotide, with short regions of DNA nucleosides, non-RNaseH recruiting oligonucleotides may be made. Advantageously, the sugar modified nucleosides are affinity enhancing sugar modified nucleosides.

Oligonucleotide mixmers are often used to provide occupation based modulation of target genes, such as splice modulators or microRNA inhibitors.

In some embodiments the sugar modified nucleosides in the mixmer, or contiguous nucleotide sequence thereof, comprise or are all LNA nucleosides, such as (S)cET or beta-D-oxy LNA nucleosides.

In some embodiments all of the sugar modified nucleosides of a mixmer comprise the same sugar modification, for example they may all be LNA nucleosides, or may all be 2′O-MOE nucleosides. In some embodiments the sugar modified nucleosides of a mixmer may be independently selected from LNA nucleosides and 2′ substituted nucleosides, such as 2′ substituted nucleoside selected from the group consisting of 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleosides. In some embodiments the oligonucleotide comprises both LNA nucleosides and 2′ substituted nucleosides, such as 2′ substituted nucleosides, such as 2′ substituted nucleoside selected from the group consisting of 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleosides. In some embodiments, the oligonucleoitide comprises LNA nucleosides and 2′-O-MOE nucleosides. In some embodiments, the oligonucleotide comprises (S)cET LNA nucleosides and 2′-O-MOE nucleosides.

In some embodiments the mixmer, or continguous nucleotide sequence thereof, comprises only LNA and DNA nucleosides, such LNA mixmer oligonucleotides which may for example be between 8-24 nucleosides in length (see for example, WO2007112754, which discloses LNA antmiR inhibitors of microRNAs).

Various mixmer compounds are highly effective as therapeutic oligomers, particularly when targeting microRNA (antimiRs) or as splice switching oligomers (SSOs).

In some embodiments, the mixmer comprises a motif

. . . [L]m[D]n[L]m[D]n[L]m . . . or

. . . [L]m[D]n[L]m[D]n[L]m[D]n[L]m . . . or

. . . [L]m[D]n[L]m[D]n[L]m[D]n[L]m[D]n[L]m . . . or

. . . [L]m[D]n[L]m[D]n[L]m[D]n[L]m[D]n[L]m[D]n[L]m . . . or

Wherein L represents sugar modified nucleoside such as a LNA or 2′ substituted nucleoside (e.g. 2′-O-MOE), D represents DNA nucleoside, and wherein each m is independently selected from 1-6, and each n is independently selected from 1, 2, 3 and 4, such as 1-3 or 1-2, and the . . . represent optional 5′ or 3′ terminal nucleosides (e.g. region D or D″), or the 5′ or 3′ terminus of the oligonucleotide, or contiguous nucleotide sequence thereof.

In some embodiments each L is a LNA nucleoside. In some embodiments, at least one L is a LNA nucleoside and at least one L is a 2′-O-MOE nucleoside. In some embodiments, each L is independently selected from LNA and 2′-O-MOE nucleoside.

In some embodiments, the mixmer may comprise or consist of a contiguous nucleotide sequence of between 10 and 24 nucleotides, such as 11, 12, 13, 14, 15, 16, 17, 18, 19, 20, 21, 22 or 23 nucleotides.

In some embodiments, the contiguous nucleotide sequence of the mixmer comprises of at least 30%, such as at least 40%, such as at least 50% LNA units.

In some embodiments, the mixmer comprises or consists of a contiguous nucleotide sequence of repeating pattern of nucleotide analogue and naturally occurring nucleotides, or one type of nucleotide analogue and a second type of nucleotide analogues. The repeating pattern, may, for instance be every second or every third nucleotide is a nucleotide analogue, such as LNA, and the remaining nucleotides are naturally occurring nucleotides, such as DNA, or are a 2′substituted nucleotide analogue such as 2′MOE of 2′fluoro analogues as referred to herein, or, in some embodiments selected form the groups of nucleotide analogues referred to herein. It is recognised that the repeating pattern of nucleotide analogues, such as LNA units, may be combined with nucleotide analogues at fixed positions—e.g. at the 5′ or 3′ termini.

In some embodiments the first nucleotide of the oligomer, counting from the 3′ end, is a nucleotide analogue, such as an LNA nucleotide or a 2′-O-MOE nucleoside.

In some embodiments, which maybe the same or different, the second nucleotide of the oligomer, counting from the 3′ end, is a nucleotide analogue, such as an LNA nucleotide or a 2′-O-MOE nucleoside.

In some embodiments, which maybe the same or different, the 5′ terminal of the oligomer is a nucleotide analogue, such as an LNA nucleotide or a 2′-O-MOE nucleoside.

In some embodiments, the mixmer comprises at least a region consisting of at least two consecutive nucleotide analogue units, such as at least two consecutive LNA units.

In some embodiments, the mixmer comprises at least a region consisting of at least three consecutive nucleotide analogue units, such as at least three consecutive LNA units.

Region D′ or D″ in an Oligonucleotide

The oligonucleotide of the invention may in some embodiments comprise or consist of the contiguous nucleotide sequence of the oligonucleotide which is complementary to the target nucleic acid, such as the gapmer F-G-F′, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein.

The addition of region D′ or D″ may be used for the purpose of joining the contiguous nucleotide sequence, such as the gapmer, to a conjugate moiety or another functional group. When used for joining the contiguous nucleotide sequence with a conjugate moiety is can serve as a biocleavable linker. Alternatively it may be used to provide exonucleoase protection or for ease of synthesis or manufacture.

Region D′ and D″ can be attached to the 5′ end of region F or the 3′ end of region F′, respectively to generate designs of the following formulas D′-F-G-F′, F-G-F′-D″ or D′-F-G-F′-D“. In this instance the F-G-F” is the gapmer portion of the oligonucleotide and region D′ or D″ constitute a separate part of the oligonucleotide.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs (e.g. gapmer regions) within a single oligonucleotide.

In one embodiment the oligonucleotide of the invention comprises a region D′ and/or D″ in addition to the contiguous nucleotide sequence which constitute the gapmer.

In some embodiments, the oligonucleotide of the present invention can be represented by the following formulae:

F-G-F′; in particular F₁₋₈-G₅₋₁₆-F′₂₋₈

D′-F-G-F′, in particular D′₁₋₃-F₁₋₈-G₅₋₁₆-F′₂₋₈

F-G-F′-D″, in particular F₁₋₈-G₅₋₁₆-F′₂₋₈-D″₁₋₃

D′-F-G-F′-D″, in particular D′₁₋₃-F₁₋₈-G₅₋₁₆-F′₂₋₈-D″₁₋₃

In some embodiments the internucleoside linkage positioned between region D′ and region F is a phosphodiester linkage. In some embodiments the internucleoside linkage positioned between region F′ and region D″ is a phosphodiester linkage.

Conjugate

The term conjugate as used herein refers to an oligonucleotide which is covalently linked to a non-nucleotide moiety (conjugate moiety or region C or third region).

Conjugation of the oligonucleotide of the invention to one or more non-nucleotide moieties may improve the pharmacology of the oligonucleotide, e.g. by affecting the activity, cellular distribution, cellular uptake or stability of the oligonucleotide. In some embodiments the conjugate moiety modify or enhance the pharmacokinetic properties of the oligonucleotide by improving cellular distribution, bioavailability, metabolism, excretion, permeability, and/or cellular uptake of the oligonucleotide. In particular the conjugate may target the oligonucleotide to a specific organ, tissue or cell type and thereby enhance the effectiveness of the oligonucleotide in that organ, tissue or cell type. A the same time the conjugate may serve to reduce activity of the oligonucleotide in non-target cell types, tissues or organs, e.g. off target activity or activity in non-target cell types, tissues or organs. WO 93/07883 and WO2013/033230 provides suitable conjugate moieties, which are hereby incorporated by reference. Further suitable conjugate moieties are those capable of binding to the asialoglycoprotein receptor (ASGPr). In particular tri-valent N-acetylgalactosamine conjugate moieties are suitable for binding to the the ASGPr, see for example WO 2014/076196, WO 2014/207232 and WO 2014/179620 (hereby incorporated by reference).

Oligonucleotide conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103, each of which is incorporated herein by reference in its entirety.

In an embodiment, the non-nucleotide moiety (conjugate moiety) is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids) or combinations thereof.

Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety (Region C), to a first region, e.g. an oligonucleotide or contiguous nucleotide sequence complementary to the target nucleic acid (region A).

In some embodiments of the invention the conjugate or oligonucleotide conjugate of the invention may optionally, comprise a linker region (second region or region B and/or region Y) which is positioned between the oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In a preferred embodiment the nuclease susceptible linker comprises between 1 and 10 nucleosides, such as 1, 2, 3, 4, 5, 6, 7, 8, 9 or 10 nucleosides, more preferably between 2 and 6 nucleosides and most preferably between 2 and 4 linked nucleosides comprising at least two consecutive phosphodiester linkages, such as at least 3 or 4 or 5 consecutive phosphodiester linkages. Preferably the nucleosides are DNA or RNA. Phosphodiester containing biocleavable linkers are described in more detail in WO 2014/076195 (hereby incorporated by reference).

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups The oligonucleotide conjugates of the present invention can be constructed of the following regional elements A-C, A-B-C, A-B-Y-C, A-Y-B-C or A-Y-C. In some embodiments the linker (region Y) is an amino alkyl, such as a C2-C36 amino alkyl group, including, for example C6 to C12 amino alkyl groups. In a preferred embodiment the linker (region Y) is a C6 amino alkyl group.

### EXAMPLES

### Example 1

Synthesis of DNA 3′-O-oxazaphospholidine monomers was performed as previously described (Oka et al., J. Am. Chem. Soc. 2008 130: 16031-16037, and Wan et al., NAR 2014, November, online publication). Synthesis of LNA monomers was performed as previously described (WO2016/079181).

### Example 2 Development of Sub-Library Discovery Method

Parent Compound:

5′-Gₛ^{m}Cₛ aₛ aₛ gₛ cₛ aₛ tₛ cₛ cₛ tₛ Gₛ T-3′ (SEQ ID NO 1) wherein capital letters represent a beta-D-oxy LNA nucleoside (2′-O—CH2-4′ bridged nucleoside in the beta-D-orientation), lowercase letters represent a DNA nucleoside, subscript s represents a stereorandom phosphorothioate linkage, and ^{m}C is 5 methyl cytosine.

Assay System:

The oligonucleotides were tested in vitro by introduction in to HeLa cells via gymnotic delivery at 5 μM concentration. Cells were harvested after 3 days.

Analysis:

Hif-1α mRNA knockdown was analyzed by qPCR.

The 13mer parent compound has 12 stereounspecified phosphorothioate internucleoside linkages. In order to identify stereodefined variants of the parent compound, two alternative approaches were utilized:

Strategy 1: 236 fully stereodefined compounds based on the parent compound were synthesized with a randomized stereodefined motif. These were screened in the assay system. The results are shown in FIG. 8. The three most potent compound identified were:

RTR34818: 5′-G_{srP} ^{m}C_{ssP} a_{ssP} a_{srP} g_{srP} c_{ssP} a_{srP} t_{srP} c_{ssP} c_{srP} t_{ssP} G_{ssP} T-3

RTR34887: 5′-G_{srP} ^{m}C_{ssP} a_{srP} a_{srP} g_{srP} c_{ssP} a_{ssP} t_{srP} c_{ssP} c_{srP} t_{ssP} G_{ssP} T-3

RTR34593: 5′-G_{srP} ^{m}C_{ssP} a_{srP} a_{srP} g_{srP} c_{ssP} a_{ssP} t_{srP} c_{srP} c_{ssP} t_{srP} G_{ssP} T-3

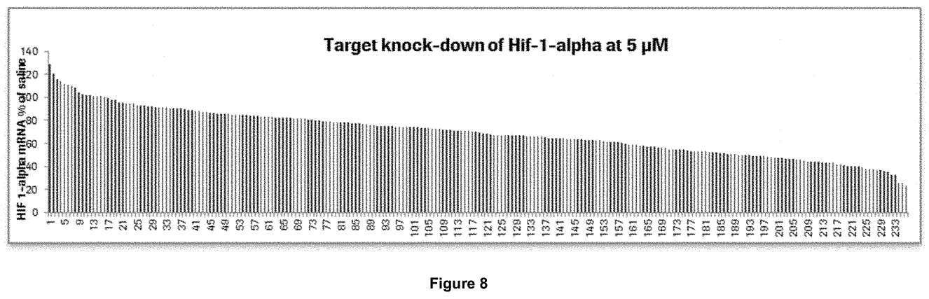

Strategy 2—Part 1: We divided the parent compound into three regions, each comprising 4 consecutive phosphorthioate linkages. For each region we made 16 sub-libraries where the phosphorothioate internucleoside linkages within the region each had 1 of the 16 possible (24) stereodefined motifs, where the remaining internucleoside linkages were stereorandom internucleoside linkages. The total number of partially stereodefined compound synthesized was therefore 16+16+16=48 sub-library compound (see FIG. 2 for a diagrammatic representation of the experiment). Each sub-library was screening in the assay system. The results are shown in FIGS. 9a, 9b and 9c.

Strategy 2—Part 1: From part 1, we identified the most potent sub-library stereodefined motif for each of the three regions and designed a fully stereodefined compound incorporating the stereodefined motif from all three most potent sub-libraries, one for each of the three regions.

The compound identified was:

RTR34593: 5′-G_{srP} ^{m}C_{ssP} a_{srP} a_{srP} g_{srP} c_{ssP} a_{ssP} t_{srP} c_{srP} c_{ssP} t_{srP} G_{ssP} T-3′

This was identical to the most potent compound identified by strategy 1, validating the sub-library approach as a method of selecting preferred optimised stereodefined variants of parent oligonucleotides without synthesising extensive libraries of individual variants. The method of the invention therefore allows for efficient discovery of stereodefined variants (either sub-libraries or full stereodefined compounds) by greatly reducing the complexity of the library of diastereoisomers. For example the position 5 RSSR sublibrary reduces the complexity of the library from 2\hat{ }4096 to 2\hat{ }8=256 diastereoisomers, and utilizing the combined sub-library approach (part 2), the complexity can be reduced from 4096 to 49.

### Example 3: Investigation of the Position Requirements for the “RSSR” Motif

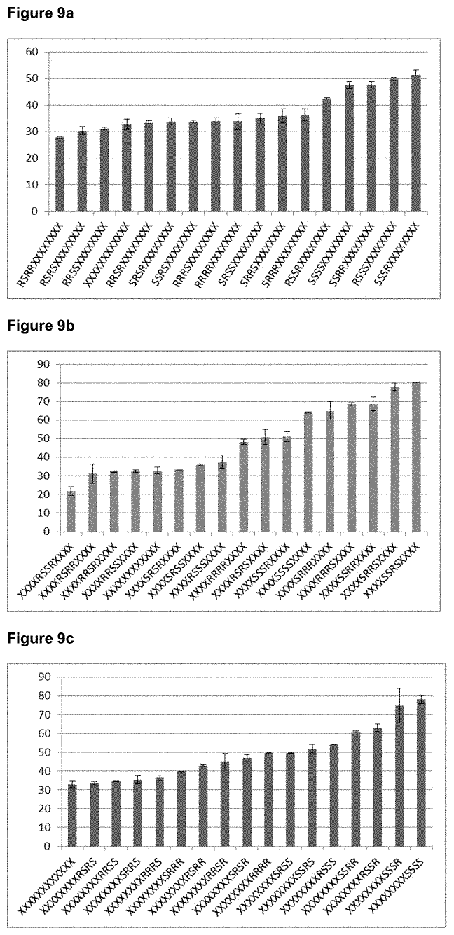

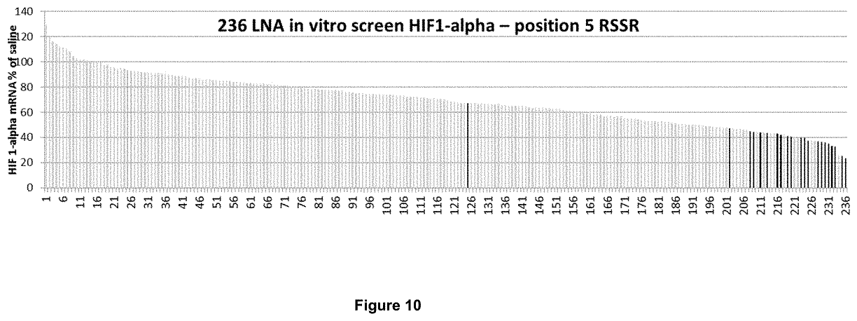

In example 2, we identified that several of the most potent sub-libraries and most potent compounds had a motif of stereodefined internucleoside linkages “5′-RSSR 3′”, positioned with the first Rp internucleoside linkage placed between the 5^{th} and 6^{th} nucleosides, refered to as position 5 (illustrated in FIG. 10). The data for the position 5-8 region sub-libraries is provided below:

| | | Average mRNA | std. |
|---|---|---|---|
| Compound | Stereomotif | knockdown | error |
| RTR48069 | XXXXSRRSXXXXH | 69.4 | 1.4 |
| RTR48070 | XXXXSSRRXXXXH | 61.1 | 2.6 |
| RTR48071 | XXXXRSSRXXXXH | 19.5 | 1.7 |
| RTR48072 | XXXXRRSRXXXXH | 28.7 | 0.2 |
| RTR48073 | XXXXSSRSXXXXH | 71.5 | 0.2 |
| RTR48074 | XXXXRRSSXXXXH | 28.9 | 0.4 |
| RTR48075 | XXXXSRRRXXXXH | 57.8 | 3.4 |
| RTR48076 | XXXXSRSSXXXXH | 32.0 | 0.2 |
| RTR48077 | XXXXSSSSXXXXH | 57.0 | 0.1 |
| RTR48078 | XXXXSSSRXXXXH | 45.5 | 1.8 |
| RTR48079 | XXXXRSSSXXXXH | 33.6 | 2.5 |
| RTR48080 | XXXXRSRRXXXXH | 27.7 | 3.6 |
| RTR48081 | XXXXRSRSXXXXH | 45.3 | 2.9 |
| RTR48082 | XXXXSRSRXXXXH | 29.6 | N/A |
| RTR48083 | XXXXRRRRXXXXH | 42.9 | 1.0 |
| RTR48084 | XXXXRRRSXXXXH | 61.0 | 0.5 |
| Parent (RTR4358) | XXXXXXXXXXXXH | 29.2 | 1.0 |

See the data for the full stereodefined compounds from strategy 1 in the tables below: Position 5 (5-8 stereodefined)

| RTR nu. | mRNA % | Chiral sequence |
|---|---|---|
| 34896 | 67.2 | SSSSRSSRRSSS |
| 34614 | 47.2 | SRRSRSSRRSSR |
| 34553 | 45 | SRRSRSSRRSSS |
| 34866 | 44.3 | SSSSRSSRSSSR |
| 34869 | 44 | RRSSRSSRRRRS |
| 34508 | 43 | SSRSRSSRRSSS |
| 34563 | 42 | RRRSRSSRSRSR |
| 34901 | 40.4 | SRRSRSSRRSRS |
| 34652 | 39.7 | RRRSRSSRRSSR |
| 34891 | 39.5 | SSRSRSSRRSRR |
| 34587 | 37.5 | RRRSRSSRSRRS |
| 25859 | 36.7 | SRSSRSSRSRSS |
| 34648 | 36.4 | SRRRRSSRSRRR |
| 34556 | 36 | SSRSRSSRSRRS |
| 34835 | 34.9 | RRSRRSSRSSSS |
| 34613 | 33 | RSSRRSSRSSRR |
| 34836 | 32.6 | SRRSRSSRRRSS |
| 34593 | 25.1 | RSRRRSSRRSRS |
| 34887 | 23.3 | RSRRRSSRSRSS |

Position 6 (6-9 Stereodefined)

| RTR nu. | mRNA % | Chiral sequense |
|---|---|---|
| 34608-1 | 111.1 | RRRRSRSSRSSR |
| 34674-1 | 102.6 | SRRRRRSSRSSS |
| 34664-1 | 83.2 | RRRSSRSSRSSS |
| 34509-1 | 77.9 | SSSRRRSSRRSR |
| 34636-1 | 73 | SSSRRRSSRRSS |
| 34813-1 | 71.2 | SRRRSRSSRRSS |
| 34875-1 | 71 | SSRSRRSSRRSS |
| 34812-1 | 66.5 | RRSSSRSSRRSR |
| 34547-1 | 63.5 | SRSSRRSSRRRR |
| 34881-1 | 54.8 | SRRSRRSSRSRS |
| 34905-1 | 52.3 | RRRSRRSSRRSS |
| 34629-1 | 52.2 | RRSSRRSSRSRS |
| 34867-1 | 51.5 | RSSRSRSSRRSS |
| 34834-1 | 45.8 | RRSRSRSSRSRS |

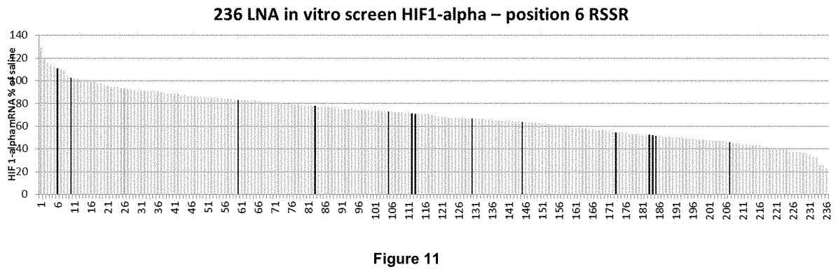

We concluded that the position of the RSSR motif was crucial to its effect on potency and than by shifting the RSSR motif 1 position, e.g. to position 6, typically resulted in a net loss of potency (also shown in FIG. 11). It was therefore concluded that the RSSR stereodefined motif is not portable within an oligonucleotide sequence.

### Example 4: In Vitro→In Vivo Translatability of the Position 5 RSSR Motif

In order to determine whether the potency enhancement of the RSSR position 5 motif in the compound of SEQ ID NO 1 was translated from in vitro to in vivo. Two stereodefined Hif1 a compounds were selected for this study:

| #18 | SRSSRSSRSRSS | GCaagcatcctGT | 5′- G_{Ssp} ^{m}C_{Srp} a_{Ssp} a_{Ssp} g_{Srp} c_{Ssp} a_{Ssp} |
|---|---|---|---|
| | | | t_{Srp} c_{Ssp} c_{Srp} t_{Ssp} G_{Ssp} T -3′ |
| #21 | SRRSSRSSRRSR | GCaagcatcctGT | 5′- G_{Ssp} ^{m}C_{Srp} a_{Srp} a_{Ssp} g_{Ssp} c_{Srp} a_{Ssp} |
| | | | t_{Ssp} c_{Srp cSrp} t_{Ssp} G_{Srp} T -3′ |

Black 6 mice were subjected to the stereodefined LNA oligonucleotide or control LNA compound mixture and the knock down of Hif-1α mRNA, the tissue content of the oligonucleotides, and ALT was measured.

Female C57BL6/J mice (5/group appr. 20 g at arrival) were injected iv with a single dose saline or 10 mg/kg LNA-antisense oligonucleotide phosphorthioate random mixture (parent from example 2) or with 10 mg/kg of stereodefined LNA antisense oligonucleotide (ID #22 or ID #18). The animals were sacrificed at day 3 and total serum was collected as well as liver and kidney.

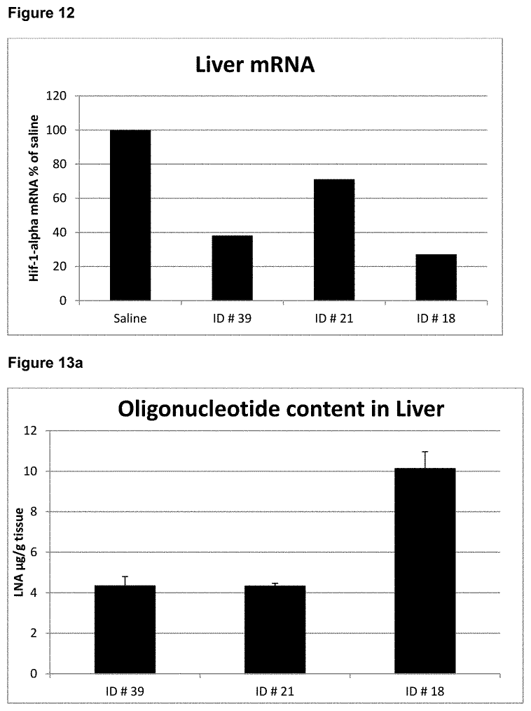

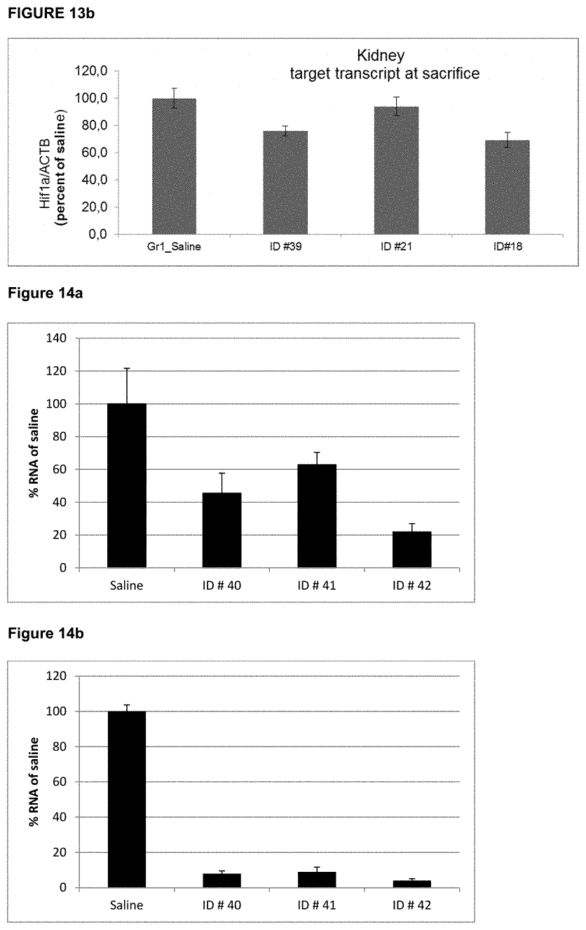

Hif-1α mRNA knockdown was analyzed by qPCR. In brief, RNA was isolated from homogenized liver and kidney using MagnaPure RNA Isolation and purification system (catalog #03604721001 and #05467535001; Roche) according to the manufacturer's instructions. RT-QPCR was done using Taqman Fast Universal PCR Master Mix 2x (Applied Biosystems Cat #4364103) and Taqman gene expression assay (mHif-1α, Mm004688869_m1 and mGAPDH #4352339E) following the manufacturers protocol. The results are shown in FIG. 12. The oligonucleotide content in the liver and kidney was measured using sandwich ELISA method and results are shown in FIGS. 13a and 13b.

### Conclusions

In the liver, the position 5 RSSR compound, RTR25859 stereodefined LNA oligonucleotides (ID #18) showed an improved effect on the mRNA target compared to the random mixture (ID #39) whereas the position 6 RSSR stereodefined LNA oligonucleotide (ID #21) showed lower down regulation of the targeted mRNA compared to the random mixture.

Notably, the tissue content in liver and kidney was higher for RTR25859 (ID #18) compared to both the random mixture (ID #39) and the other stereodefined version (ID #21). Those two (ID #39 and ID #21) have similar uptake in liver but the stereodefined LNA (ID #21) has lover uptake in kidney tissue compared to both the random mixture (ID #39) and ID #18. The stereodefined LNA's (ID #18 and ID #21) have different uptake and potency compared to the random mixture (ID #39) as well as compared to each other. This example illustrates that the preferred motif identified is translatable between in vitro and in vivo experiments, and that potency may be related to enhanced uptake.

### Example 5 In Vivo Effect on ApoB mRNA of Stereodefined LNA Oligonucleotides Versus the Random Mixture LNA

Having illustrated that for a single compound, the position 5 RSSR motif was a preferred motif in vitro and in vivo (Examples 3 & 4), we wished to determine whether the motif was transferable between antisense oligonucleotides of different sequences.

Parent oligonucleotide: (#40) GₛⁿCₛaₛtₛtₛgₛgₛtₛaₛtₛTₛ^{m}CₛA, (SEQ ID NO 2) wherein capital letters represent a beta-D-oxy LNA nucleoside (2′-O—CH2-4′ bridged nucleoside in the beta-D-orientation), lower case letters represent a DNA nucleoside, subscript s represents a stereorandom phosphorothioate linkage, and ^{m}C is 5 methyl cytosine.

Stereodefined variants used:

| | | | |
|---|---|---|---|
| #41 | SRRSSRSSRRSR | GCattggtatTCA | 5′- G_{Ssp} ^{m}C_{Srp} a_{Srp} t_{Ssp} t_{Ssp} g_{Srp} g_{Ssp} t_{Ssp} |
| | | | a_{Srp} t_{Srp} T_{Ssp} ^{m}C_{Srp} A -3′ |
| #42 | RRSSRSSRSRSS | GCattggtatTCA | 5′- G_{Srp} ^{m}C_{Srp} a_{Ssp} t_{Ssp} t_{Srp} g_{Ssp} g_{Ssp} t_{Srp} |
| | | | a_{Ssp} t_{Srp} T_{Ssp} ^{m}C_{Ssp} A -3′ |

Black 6 mice were subjected to the stereodefined LNA oligonucleotide or control LNA compound mixture and the knock down of ApoB mRNA, the tissue content of the oligonucleotides, ALT, and total cholesterol was measured.

Female C57BL6/J mice (5/group appr. 20 g at arrival) were injected iv with a single dose saline or 1 mg/kg LNA-antisense oligonucleotide phosphorthioate random mixture (ID #40) or with 1 mg/kg of stereodefined LNA antisense oligonucleotide (ID #41 or ID #42 of the invention). Blood samples of 50 μl were collected pre-dosing at day minus 6, and post dosing at day 3. The animals were sacrificed at day 7 and total serum was collected as well as liver and kidney. ApoB mRNA knockdown was analyzed by qPCR. In brief, RNA was isolated from homogenized liver and kidney using MagnaPure RNA Isolation and purification system (catalog #03604721001 and #05467535001; Roche) according to the manufacturer's instructions. RT-QPCR was done using Taqman Fast Universal PCR Master Mix 2x (Applied Biosystems Cat #4364103) and Taqman gene expression assay (mApoB, Mm01545150_m1 and mGAPDH #4352339E) following the manufacturers protocol. The results are shown in FIG. 14a and FIG. 14b.

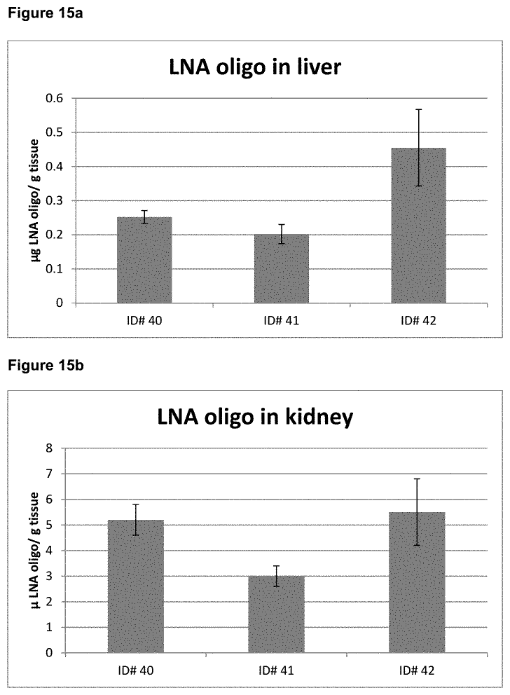

The oligonucleotide content in the liver and kidney was measured using sandwich ELISA method and results are shown in FIG. 15a and FIG. 15b.

Sampling of Liver and Kidney Tissue.

The animals were anaesthetized with 70% CO₂-30% O₂ and sacrificed by cervical dislocation at day 7 for the ApoB target. One half of the large liver lobe and one kidney were minced and submerged in RNAlater. The other half of liver and the other kidney was frozen and used for tissue analysis. Oligonucleotide content in liver and kidney was measured by sandwich ELISA method (essentially as described in Lindholm et al, Mol Ther. 2012 February; 20(2):376-81).

Total cholesterol in serum was measured using ABX Pentra Cholesterol CP (Triolab, Brondby, Denmark) according to the manufacturer's instructions. The results are shown in FIG. 16.

### Conclusions

In the liver one of the stereodefined LNA oligonucleotides (ID #42) showed a remarkable improved effect on the mRNA target compared to the random mixture (ID #40) whereas the other stereodefined LNA oligonucleotide (ID #41) showed similar effect on the targeted mRNA compared to the random mixture. The total cholesterol readout supports the mRNA effect in that stereodefined LNA oligonucleotide ID #42 is far more potent than the random mixture ID #40 and the other stereodefined version ID #41. The results indicate that for optimal in vivo efficacy, the RSSR motif should also be placed at position 5 (as seen with the Hif1alpha compound) and that a single shift of the motif towards the 3′ end resulted in compound which was less potent than the non stereodefined control.

We have previously reported on markedly different RNaseH activity of ApoB compounds based on the parent compound #40, and review of that data failed to identify RSSR position 5 as correlated to an increaded RNaseH activity: See example 7 in WO2016/096938. We therefore conclude that the enhanced potency of position 5 RSSR compound is not due to an enzymatic preference of the RNaseH enzyme.

### Example 6 Testing In Vitro Efficacy of Oligonucleotides Targeting a Human mRNA, in U251 Cell Line at Single Dose Concentration

In order to validate whether the position 5 RSSR motif was potable to a different LNA gapmer targeting a different target and of different sequence and design, we made 263 fully stereorandom variants of an LNA Gapmer compound of design: 5′ LLLdddddddddLLLL 3′ wherein L is a beta-D-oxy LNA nucleoside (2′-O—CH₂-4′ bridged nucleoside in the beta-D-orientation), and d represent a DNA nucleoside, wherein all internucleoside linkages are stereodefined phosphorothioate internucleoside linkages.

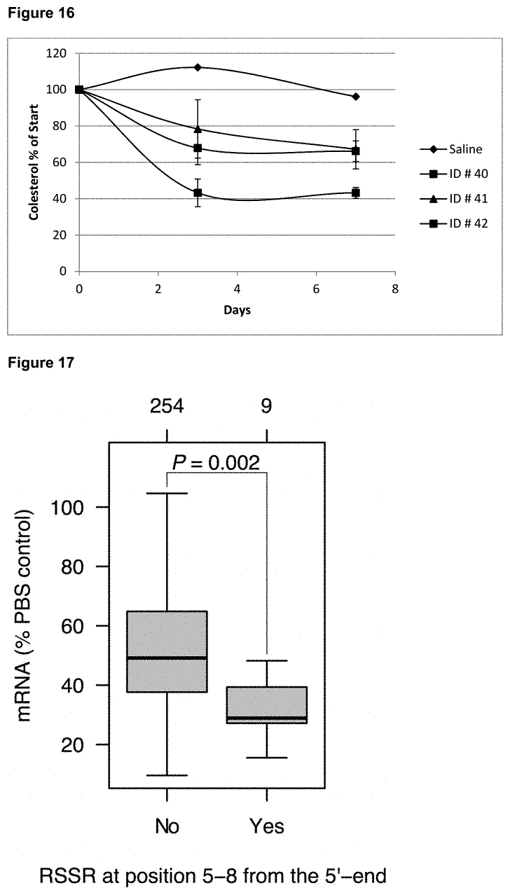

Human glioblastoma U251 cell line was purchased from ECACC and maintained as recommended by the supplier in a humidified incubator at 37° C. with 5% CO₂. For assays, 2000 U251 cells/well were seeded in a 96 multi well plate in media recommended by the supplier. Cells were incubated for 2 hours before addition of oligonucleotides dissolved in PBS. Concentration of oligonucleotides: 5 μM. 4 days after addition of oligonucleotides, the cells were harvested. RNA was extracted using the PureLink Pro 96 RNA Purification kit (Ambion, according to the manufacturer's instructions). cDNA synthesis and qPCR were performed using qScript XLT one-step RT-qPCR ToughMix Low ROX, 95134-100 (Quanta Biosciences). TaqMan primer assays were used to detect the target mRNA and house keeping gene, GAPDH. All primer sets were purchased from Life Technologies The relative expression of the target mRNA expression level in the table is shown as % of control (PBS-treated cells). The results are shown in FIG. 17. As with the 13mer Hif1alpha 2-9-3 compound, and the 2-8-3 ApoB compound, the 16mer 3-9-4 compounds with position 5 RSSR motif were significantly more potent.

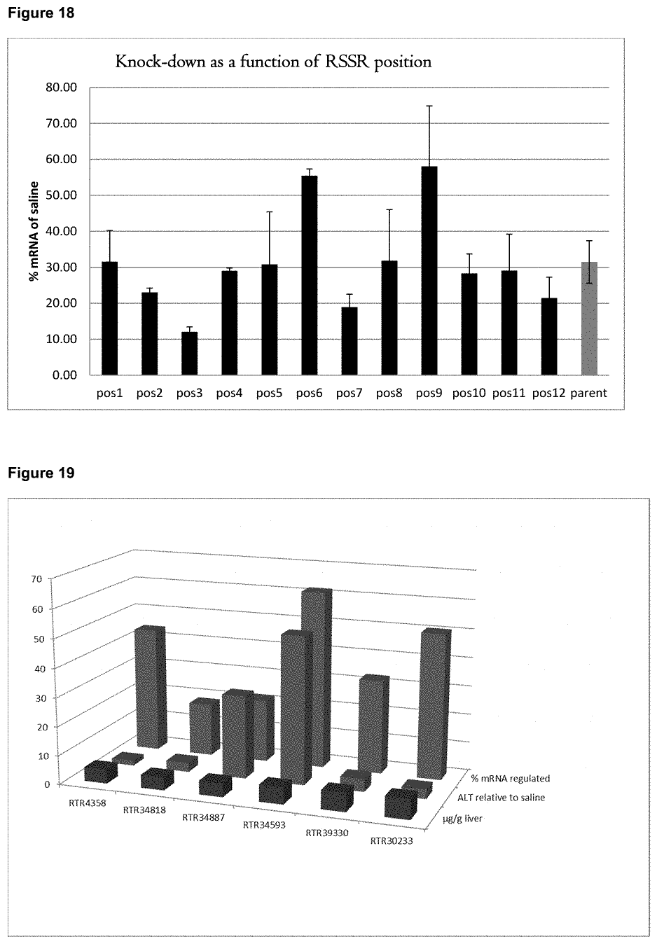

In order to further validate this, we repeated the experiment using an independent target and sequence, this time using the sub-library approach to walk the RSSR motif through a 13 nucleotide parent LNA gapmer oligonucleotide of design (a motif walk approach). Contrary to the result obtained from the ApoB and Hif1alpha compound, the position 5 sub-library was no more potent than that of the parent, and in this instance a psotion 3 RSSR motif was significantly more potent (FIG. 18).

We therefore conclude that whilst some stereodefined motifs may be associated with an enhanced property of a stereodefined variant, the effect of such a motif is dependent upon the context of the individual oligonucleotide, such as the sequence, chemical modification, and design of the oligonucleotide.

### Example 7 Multi-Parameter Optimisation of Stereodefined Variants

In order to determine whether we could utilise the discovery methods disclosed herein, we evaluated a range of fully stereodefined compounds identified by in vitro potency and in vitro hepatotoxicity assays (disclosed in WO2016/096938) in an in vivo experiment in mouse, and evaluated the potency, hepatotoxicity and oligo content of a selection of compounds.

The compounds used in the in vivo experiment were all based on the Hif1alpha parent compound and were as follows:

| Oligo | Chirality | Sequence |
|---|---|---|
| RTR4358 | Mix | G^{m}CaagcatccsGT |
| RTR34818 | RSSRRSRRSRSS | G^{m}CaagcatccsGT |
| RTR34887 | RSRRRSSRSRSS | G^{m}CaagcatccsGT |
| RTR34593 | RSRRRSSRRSRS | G^{m}CaagcatccsGT |
| RTR39330 | RRSSRSSRSRSS | G^{m}CaagcatccsGT |
| RTR30233 | SRRSRSSRRSRR | G^{m}CaagcatccsGT |

Compounds 34887, 34593, 39330 and 30233 all comprise a position 5 RSSR motif. Compound 34818 does not have the position 5 RSSR motif. The experiment was performed as per example 4, and the data is shown in FIG. 19.

FIG. 19 shows that whilst the RSSR position 5 motif can provide compounds with enhanced in vivo potency, there are position 5 RSSR compounds which are not as potent in vivo as the parent compounds. There is however, no correlation between potency and toxicity and as such the methods of the present invention may be used to identify compounds which have an enhanced potency without introducing a elevation of hepatotoxicity. We were also surprised to find that there was no correlation between the potency or hepatotoxicity of the tested compounds and the liver, although as with the in vivo experiments decribed in examples 4 and 5, the most potent RSSR compounds had elevated oligo content as compared to the parent compound. This results obtain illustrate the considerable in vivo pharmacological diversity created between individual fully stereodefined compounds, and the unpredictability in pharmacological performance between compounds with very similar stereodefined motifs, further highighing the value in the multiple sub-library discovery methods disclosed herein in identifying pharmacologically improved compounds.

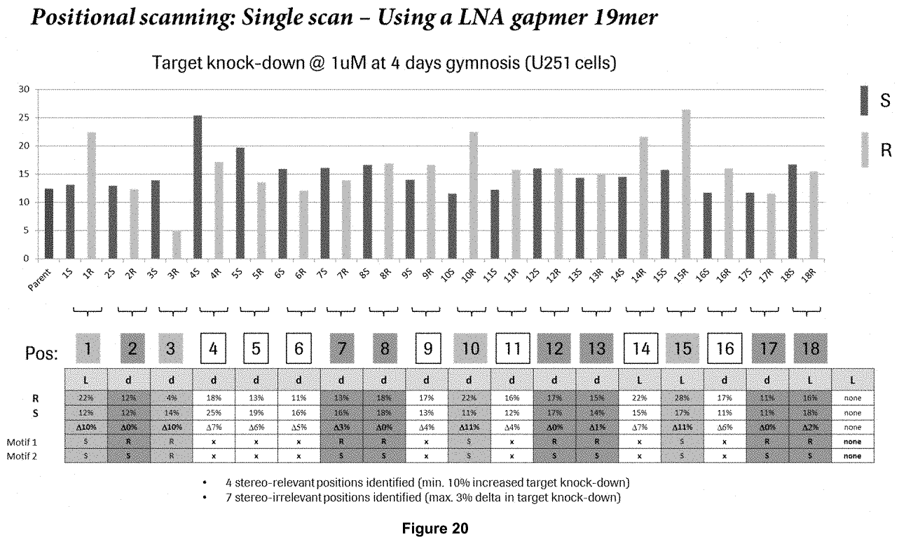

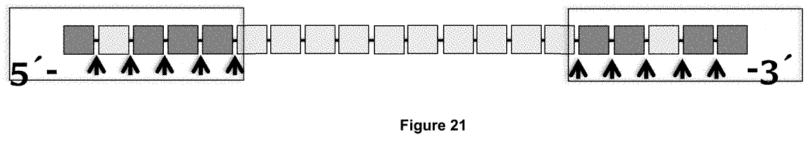

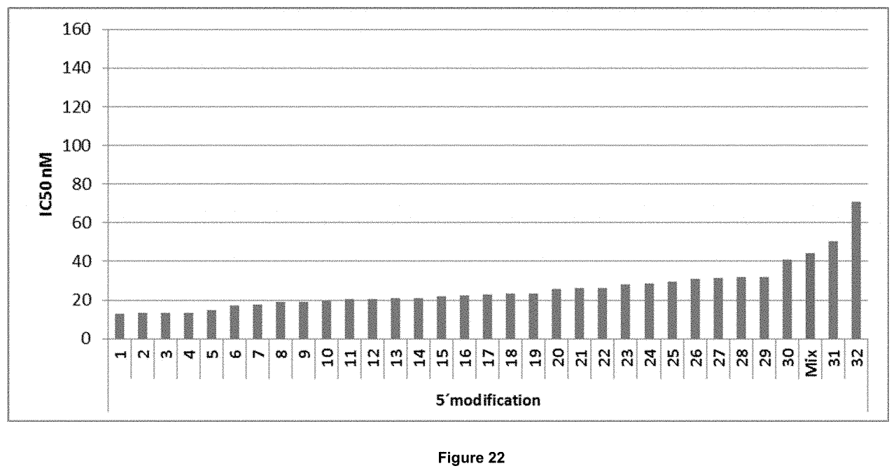

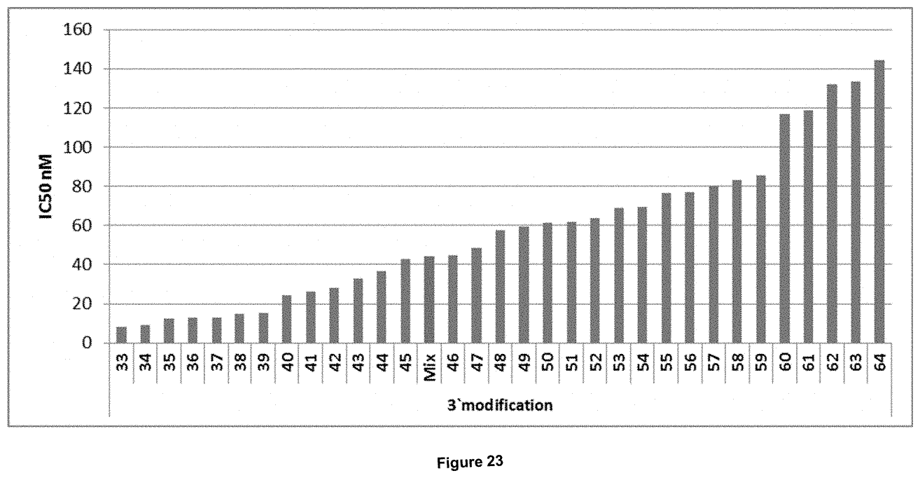

### Example 8/FIG. 20

Single position motif walk. A stereorandom 19mer LNA gapmer parent compound which was selected, and two libraries were generated, one where a single Sp stereodefined internucleoside linkage was walked across the oligonucleotide, so that each member of the library differed with respect to the position of the Sp stereodefined linkage, and a second library where a single Rp stereodefined internucleoside linkage was walked across the oligonucleotide, so that each member of the library differed with respect to the position of the Rp stereodefined linkage. In this experiment, the remaining internucleoside linkages were stereorandom. Each member of each library was assayed for potency against the mRNA target in U251 cells using gymnotic delivery of 1 μM (See example 6 for the methodology). mRNA target knock-down for each library member was determined. The results identified 4 positions where the stereodefinition (Sp or Rp) was a notable determinant of oligonucleotide potency, and 7 positions where the stereochemistry was not a relevant determinant of oligonucleotide potency. This approach allows the design of partially stereodefined compounds which comprise the preferred stereodefined internucleoside linkage at the stereo-relevant positions, and stereorandom internucleoside linkages at the stereo-irrelevant positions. Such optimized sub-library compounds may be used in further optimization methods (e.g. of the invention), to identify further stereodefined variants, including fully stereodefined variants, which have further improved properties.

The position walk experiment or methods described herein about maybe repeated using sub-libraries where the background internucleoside linkages in each library rather than being stereorandom are all either Sp or all either Rp (stereopure background linkages). For a single internucleoside linkage walk, a single Sp internucleoside linkage may be walked in a background of Rp internucleoside linkages, and a single Rp internucleoside linkage may be walked in a background of Sp internucleoside linkages.

1. A method for identifying improved stereodefined phosphorothioate variants of an antisense oligonucleotide, said method comprising the steps of:
a. Providing a parent oligonucleotide, with a defined sequence and nucleoside modification pattern;
b. Generating a library of stereodefined phosphorothioate oligonucleotides which retain the defined sequence and nucleoside modification pattern of the parent oligonucleotide,
wherein either
(i) each member of the library is a sub-library comprising a mixture of stereodefined phosphorothioate antisense oligonucleotides diastereoisomers, wherein each member of the mixture comprises a stereodefined internucleoside motif region, wherein, the stereodefined internucleoside motif region is a common region of 3-8 or 2-8 contiguous nucleosides, wherein the remaining internucleoside linkages comprise stereorandom phosphorothioate internucleoside linkages; wherein, the length and the position of each common stereodefined internucleoside linkage motif region is the same between each member of the library; and wherein, each member of the library comprises a different common stereodefined internucleoside motif in the stereodefined internucleoside motif region;
or
(ii) wherein each member of the library is a sub-library comprising a mixture of stereodefined phosphorothioate antisense oligonucleotides diastereoisomers, wherein each member of a mixture comprises a common stereodefined internucleoside linkage motif at the same position in the oligonucleotide, wherein the remaining internucleoside linkages comprise stereorandom phosphorothioate internucleoside linkages; wherein each member of the library comprises the same common stereodefined internucleoside linkage motif, wherein the position of the common stereodefined internucleoside linkage motif differs between each member of the library;
c. Screening each member of the library generated in step b) for at least one improved property, such as improved potency and/or reduced toxicity, as compared to the parent oligonucleotide;
d. Identifying one or more members of the library which have the improved property.

2. The method according to claim 1, wherein step b. comprises the step defined in step b(i).

3. The method according to claim 2, wherein, the length of each stereodefined internucleoside linkage motif region is 3, 4, 5 or 6 contiguous nucleotides (or 2, 3, 4 or 5 nucleoside linkages).

4. The method according to claim 2, wherein, the each stereodefined internucleoside linkage motif region is 3 or 4 nucleosides linkages.

5. The method according to claim 2, wherein the library comprises members of each of the possible stereodefined internucleoside linkage motifs within the stereodefined internucleoside linkage motif region.

6. The method according to claim 2, wherein each member of the library each comprises
a triplex linkage motif selected from the group consisting of RRR, RSR, RRS, RSS, SSS, SRS, SSR, and SRR, or
a quadruplex linkage motif selected from the group consisting of RRRR, RRRS, RRSR; RSRR, RRSS; RSRS; RSSR; RSSS, SSSS, SSSR; SSRS; SRSS; SSRR; SRSR; SRRS, and SRRR, or
a pentaplex linkage motif selected from the group consisting of RRRRR,RRRRS, RRRSR,RRRSS, RRSRR; RRSRS, RSRRR, RRSSR; RSRSR; RSSRR; RSSSR, SSSSR, SSSRR; SSRSR; SRSSR; SSRRR; SRSRR; SRRSR, SRRRR, RSRRS, RRSSS; RSRSS; RSSRS; RSSSS, SSSSS, SSSRS; SSRSS; SRSSS; SSRRS; SRSRS; SRRSS, and SRRRS

7. The method according to claim 2, wherein the library is comprehensive.

8. The method according to claim 2, wherein at least 30%, such as at least 40% or at least 50%, or a majority of, or all the remaining internucleoside linkages within the antisense oligonucleotide of each library member are stereorandom phosphorothioate internucleoside linkages.

9. The method according to claim 2, wherein the method further comprises the steps of
e) Selecting at least one improved oligonucleotide variant identified in step d) f) Generating a library of stereodefined phosphorothioate oligonucleotides which retain the defined sequence and nucleoside modification pattern and the same stereodefined internucleoside motif of the improved oligonucleotide variant, wherein each member of the library comprises one or more further stereodefined phosphorothioate internucleoside linkages, and wherein each member of the library differs with respect to the pattern of further stereodefined phosphorothioate internucleoside linkages,
g. Screening each member of the library generated in step f) for at least one improved property, which may be the same of different improved properties(s) as assayed in step c).

10. The method according to claim 2, wherein the step b(i) of the method comprises the generation of multiple libraries wherein each library is as defined as in step b(i) and wherein the position of each common stereodefined internucleoside linkage motif region is different between each of the multiple libraries.

11. The method according to claim 10, wherein the method further comprises the step of identifying at an improved stereodefined variants from each of the multiple libraries, and preparing a further stereodefined variant which comprises the stereodefined internucleoside linkage motifs of each of the identified improved stereodefined variants from of the multiple libraries.

12. The method according to claim 11, wherein at least two or at least three multiple libraries are screened to identify an improved stereodefined variants from each of the multiple libraries, wherein each library is as defined as in step b(i).

13. The method according to claim 12 wherein the further stereodefined variant oligonucleotide or contiguous nucleotide sequence thereof is a fully stereodefined phosphorothioate sequence.

14. The method according to claim 1, wherein step b. comprises the step defined in step b(ii).

15. The method according to claim 14, wherein the length of the common stereodefined internucleoside linkage motif is 1-6 internucleoside linkage, such as 2, 3, 4 or 5 internucleoside linkages.

16. The method according to claim 15, wherein the common stereodefined internucleoside linkage motif comprises is either
a triplex linkage motif selected from the group consisting of RRR, RSR, RRS, RSS, SSS, SRS, SSR, and SRR, or
a quadruplex linkage motif selected from the group consisting of RRRR, RRRS, RRSR; RSRR, RRSS; RSRS; RSSR; RSSS, SSSS, SSSR; SSRS; SRSS; SSRR; SRSR; SRRS, and SRRR, or
a pentaplex linkage motif selected from the group consisting of RRRRR,RRRRS, RRRSR,RRRSS, RRSRR; RRSRS, RSRRR, RRSSR; RSRSR; RSSRR; RSSSR, SSSSR, SSSRR; SSRSR; SRSSR; SSRRR; SRSRR; SRRSR, SRRRR, RSRRS, RRSSS; RSRSS; RSSRS; RSSSS, SSSSS, SSSRS; SSRSS; SRSSS; SSRRS; SRSRS; SRRSS, and SRRRS.

17. The method according to claim 14, wherein the common stereodefined internucleoside linkage motif is or comprises RSSR.

18. The method according to claim 14, wherein the library is a comprehensive oligonucleotide walk.

19. The method according to claim 1, wherein the improved property is selected from the group consisting of in enhanced or optimized affinity, enhanced stability, enhanced potency, enhanced efficacy, enhanced specific activity, reduced toxicity, altered biodistribution, enhanced cellular or tissue uptake, enhanced duration of action, and/or enhanced target specificity.

20. The method according to claim 1, wherein the improved property is assayed in vitro.

21. The method according to claim 1, wherein the antisense oligonucleotides is an RNase H recruiting oligonucleotides such as antisense oligonucleotide gapmer oligonucleotides, or is a mixmer or a totalmer.

22. The method according to claim 21, wherein the antisense oligonucleotides are LNA oligonucleotides, such as an LNA gapmer oligonucleotide.

23. The method according to claim 14, wherein the length of the antisense oligonucleotide is 7-26 nucleotides in length, such as 12-24 nucleotides in length.

24. A LNA gapmer oligonucleotide selected from the group consisting of
(SEQ ID NO 1)
5′-G_{srP}^{m}C_{ssP}a_{ssP}a_{srP}g_{srP}c_{ssP}a_{srP}t_{srP}c_{ssP}c_{srP}t_{ssP}G_{ssP}
T-3′
or
(SEQ ID NO 1)
5′-G_{srP}^{m}C_{ssP}a_{srP}a_{srP}g_{srP}c_{ssP}a_{ssP}t_{srP}c_{ssP}c_{srP}t_{ssP}G_{ssP}
T-3′
or
(SEQ ID NO 1)
5′-G_{srP}^{m}C_{ssP}a_{srP}a_{srP}g_{srP}c_{ssP}a_{ssP}t_{srP}c_{srP}c_{ssP}t_{srP}G_{ssP}
T-3′
wherein capital letters represent a beta-D-oxy LNA nucleoside (2′-O—CH2-4′ bridged nucleoside in the beta-D-orientation), lower case letters represent a DNA nucleoside, subscript _{ssP} represents an Sp stereodefined phosphorothioate linkage, and _{srP} represents a Rp stereodefined phosphorothioate linkage. ^{m}C represents a 5-methyl cytosine LNA nucleoside, or a pharmaceutically acceptable salt thereof.

25. A conjugate comprising the LNA gapmer oligonucleotide according to claim 24, and at least one conjugate moiety covalently attached to said oligonucleotide.

26. The conjugate of claim 25, wherein the conjugate moiety is capable of binding to the asialoglycoprotein receptor, such as a GalNAc conjugate moiety.

27. A pharmaceutical composition comprising the LNA gapmer oligonucleotide according to claim 24, and a pharmaceutically acceptable diluent, carrier, salt and/or adjuvant.

28. A pharmaceutically acceptable salt of the LNA gapmer oligonucleotide according to claim 24.

29. The LNA gapmer oligonucleotide according to claim 24, for use in medicine.

30. The LNA gapmer oligonucleotide according to claim 24 for use in the treatment of cancer.

31. Use of the LNA gapmer oligonucleotide according to claim 24 for the manufacture of a medicament for treatment of cancer.
